(19) United States
(12) Patent Application Publication
(10) Pub. No.: US 2026/0294607 A1
(43) Pub. Date: Oct. 1, 2026

(54) SYSTEMS AND DEVICES FOR MEDICAMENT SEED RETENTION WITHIN THE GINGIVAL CREVICE AND METHODS THEREOF

(71) Applicant: 3i Biotech, LLC, Lubbock, TX (US)

(72) Inventors: David Syn, Lubbock, TX (US); Seth Seth, Lubbock, TX (US); Rohan Ingrole, Lubbock, TX (US)

(73) Assignee: 3i Biotech, LLC, Lubbock, TX (US)

(21) Appl. No.: 19/096,556

(22) Filed: Mar. 31, 2025

(51) Int. Cl. A61C 19/06

(52) U.S. Cl. CPC A61C 19/063; A61C 2201/00

(57) ABSTRACT

A medicament seed configured for enhanced retention within a patient's gingival crevice, and systems for delivery. The medicament seed includes a carrier carrying a medicament for release. To counteract displacement forces within the gingival crevice, the medicament seed includes retention features. The retention features may include specific geometric shapes adapted to wedge against anatomical structures like the interdental papilla, surface modifications such as microstructures, bioadhesive coatings, or moisture-activated adhesives, conformable gels or viscous fluids that adapt to the crevice shape, or using expandable materials that wedge the medicament seed in place upon expansion.

### TECHNICAL FIELD

The present disclosure relates generally to medicament delivery devices, and more particularly to devices and methods for medicament seed retention within the gingival crevice.

### BACKGROUND

Medicaments, such as micronutrient supplements, medications, vaccines, and other therapeutic substances, are essential for maintaining and enhancing human health across various populations. People depend on these medicaments for numerous purposes, ranging from preventing nutritional deficiencies to managing acute and chronic diseases. The broad spectrum of medicament types highlights not only the complexity of human health requirements but also emphasizes the need to create effective delivery systems. These systems must ensure that therapeutic substances efficiently and safely reach their intended targets within the body.

The methods of medicament delivery have evolved considerably over the years, driven by advances in pharmacology, materials science, and biomedical engineering. Traditional methods such as oral ingestion, intravenous injections, and topical applications have long been the standard. However, each approach comes with its own set of challenges. Oral delivery, for example, is convenient but can be limited by issues of bioavailability and first-pass metabolism in the liver. Parenteral routes, including injections, provide rapid systemic distribution but are often associated with pain, risk of infection, and the need for trained personnel to administer the medication. Topical applications can provide localized effects but may not penetrate deeply enough to reach systemic circulation effectively. These limitations have spurred the exploration of alternative delivery methods that can offer improved efficacy, safety, and patient compliance.

One approach is the delivery of medicaments through the gingival crevice, which includes the space between the teeth and gum tissue. More specifically, the junctional epithelium within the gingival crevice shows considerable promise as a target for medicament delivery. The junctional epithelium has a high permeability due to its unique cellular structure and close contact with the underlying connective tissues, which are rich in immune cells. Because of this anatomical arrangement, the junctional epithelium allows medicaments to be rapidly absorbed directly into systemic circulation. As a result, this route has the potential to be more effective compared to conventional delivery methods.

In some implementations, delivery of the medicament into the gingival crevice may involve a medicament seed (e.g., a specially designed medicament carrier intended for placement into the gingival crevice, specifically within the junctional epithelium). The medicament seed may be configured to dissolve gradually once deposited into the gingival crevice, releasing the medicament directly to the targeted site. By delivering the medicament into the gingival crevice via the medicament seed, this technique can maximize bioavailability while minimizing systemic side effects. The seed-like carrier structure may support a controlled and sustained release of the medicament into the gingival crevice, which may be beneficial in cases where extended exposure to the medicament is necessary. Additionally, gingival crevice delivery reduces the risk of medicament degradation that often occurs when substances encounter the harsh environment of the gastrointestinal tract or systemic circulation.

However, despite the promising potential of medicament delivery via the gingival crevice, gingival crevice delivery comes with significant challenges. One issue is the tendency of the medicament seed to become displaced after placement within the gingival crevice. This displacement occurs primarily due to the unique anatomical and environmental conditions present in the junctional epithelium and gingival crevice. For example, the junctional epithelium naturally has a V-shaped structure, narrower at the base and wider at the top. This geometric shape inherently creates a force pushing the medicament seed upward from the deeper, narrower region toward the broader, upper area. Moreover, the moist environment of the gingival crevice, continually exposed to saliva, further aggravates this displacement tendency, making it challenging to retain the seed within the junctional epithelium for dissolution and delivery of the medicament.

Medicament seed displacement is particularly concerning because it can lead to premature migration of the medicament seed before complete dissolution and medicament release. If the medicament seed does not remain in position within the junctional epithelium for the necessary duration, the medicament may not be effectively absorbed, which may reduce its clinical effectiveness. Thus, securely maintaining the medicament seed in place within the gingival crevice, specifically within the junctional epithelium, is crucial for the success of this delivery strategy.

### BRIEF SUMMARY

The present disclosure relates to systems, devices, and methods configured to enhance retention of medicaments deposited into the gingival crevice and/or the junctional epithelium within the gingival crevice. Various embodiments described herein provide a medicament seed that is configured to provide therapeutic substances, such as micronutrient supplements, medications, vaccines, and other beneficial compounds, and to enhance the retention of the medicament seed within the gingival crevice to ensure that the medicament seed stays securely positioned in the gingival crevice long enough for adequate release of the medicament into the gingival crevice. In this manner, the medicament seed of embodiments is configured to overcome these inherent challenges with retention of the medicament seed within the gingival crevice due to the unique anatomical structure of the gingival crevice (and the junctional epithelium) and the dynamic, moist conditions within the gingival crevice, resulting in improved bioavailability and quicker absorption directly into systemic circulation.

In embodiments, the medicament seed may be configured with a surface configured to mechanically engage with the surrounding tissue of the gingival crevice. The surface of the medicament seed of embodiments may include various microstructural features, such as texturing, micro-barbs, ridges, grooves, etc., that may be configured to operate to anchor the medicament seed within the gingival crevice. In additional or alternative embodiments, the surface of the medicament may include bioadhesive or bioresorbable materials, which may promote adhesion to the soft tissue of the junctional epithelium. In additional or alternative embodiments, the medicament seed may be coated with an adhesive substance or a moisture-activated glue that, upon exposure to the moist environment within the gingival crevice, is activated to secure the medicament seed in place. These retention features may ensure that the medicament seed remains in position until it dissolves or degrades sufficiently to release the medicament.

In embodiments, the medicament seed may be configured with a variety of geometric configurations and shapes configured to enhance retention. For example, the medicament seed may be configured with a tapered, stepped, or bulbous shape to create resistance against displacement once positioned within the gingival crevice. In another embodiment, the medicament seed may be configured as an A-frame structure having two legs connected at a vertex. This A-frame may be configured to conform to the inverted V-shaped interdental papilla (e.g., the vertex of the A-frame may be configured to abut or wedge against the apex of the interdental papilla while the legs extend within the gingival crevice). This configuration may counter the natural preferential motion (e.g., induced by the shape of the gingival crevice and the presence of saliva) that might otherwise cause the medicament seed to migrate from the deeper regions toward the opening of the gingival crevice. In some embodiments, the A-frame configuration may be further modified so that one of the legs of the A-frame is shorter than the other, or even a single leg, which may simplify the design and may reduce material usage while still enabling the vertex of the A-frame to wedge within the interdental papilla.

In embodiments, the medicament seed may be configured with a material consistency configured to promote retention by conforming or adapting to the shape of the gingival crevice. For example, the medicament seed may be formulated as a gel or highly viscous fluid that, upon deposition into the gingival crevice, slowly flows to fill the gingival crevice and conform to the gingival crevice's contours. This formulation may then harden, either partially or fully, ensuring that the medicament seed remains in place until sufficient dissolution has occurred. In yet another embodiment, the medicament seed may comprise a sponge-like material that expands when exposed to the moist environment within the gingival crevice. The expansion may generate a wedging effect as the sponge-like material presses against the walls of the gingival crevice and the junctional epithelium, which may enhance retention through frictional engagement and adhesion.

In this manner, the embodiments described herein address the significant challenge of premature displacement of the medicament seed due to the anatomical shape and moist conditions of the gingival crevice. By incorporating features disclosed herein, the present disclosure provides a robust solution for the controlled and sustained delivery of medicaments into the gingival crevice. This innovative approach not only ensures that the medicament is released in a controlled manner but also maximizes therapeutic outcomes by delivering the medicament directly to the junctional epithelium for rapid absorption into the patient's system.

As such, the present disclosure provides a comprehensive system for medicament delivery that utilizes a medicament seed configured for enhanced retention within the gingival crevice and/or the junctional epithelium. The embodiments disclosed herein ensure that the medicament seed remains securely in place until the therapeutic agent is effectively released. This retention-focused configuration overcomes the challenges inherent in traditional medicament delivery methods, providing improved bioavailability, enhanced patient outcomes, and a versatile platform for the treatment of a wide array of health conditions.

It is an object of the disclosure to provide a medicament seed configured for enhanced retention within a gingival crevice of a patient. It is a further object of the disclosure to provide a system for delivering medicament into one or more gingival crevices of a patient.

In one particular embodiment, a medicament seed configured for enhanced retention within a gingival crevice of a patient is provided. The medicament seed includes a carrier configured to be deposited into the gingival crevice and to carry a medicament for release within the gingival crevice to be absorbed into the patient's system through a junctional epithelium, and a retention feature integrated with the carrier. In embodiments, the retention feature is configured to interact with one or more anatomical structures of an oral cavity of the patient to resist displacement forces within the gingival crevice and maintain the carrier within the gingival crevice for a period sufficient for the release of the medicament into the gingival crevice.

In another embodiment, a system for delivering medicament into one or more gingival crevices of a patient is provided. The system includes a medicament seed comprising a carrier configured to carry a medicament and a retention feature configured to resist displacement forces within the gingival crevice, and a deposition device configured to facilitate placement of the medicament seed into the one or more gingival crevices.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

FIG. 1A shows a perspective view of a portion of an oral cavity illustrating a representative gingival crevice adjacent to a tooth.

FIGS. 1B-1E show exemplary anatomy of a gingival crevice in the gingiva for targeting deposition of medicament in accordance with embodiments of the present disclosure.

FIGS. 1F-1H shows various examples of medicament seed insertion into the gingival crevice in accordance with embodiments of the present disclosure.

FIG. 1I shows a top view of the exemplary anatomy of the gingival crevice in the gingiva for targeting deposition of medicament in accordance with embodiments of the present disclosure.

FIG. 1E illustrates a front view of the gum pocket anatomy of a patient that may be targeted for medicament deposition in accordance with embodiments of the present disclosure.

FIG. 2A illustrates medicament seed configured for enhanced retention within the gingival crevice in accordance with embodiments of the present disclosure.

FIG. 2B illustrates the medicament seed 210 after deposition into one or more gingival crevices in accordance with embodiments of the present disclosure.

FIG. 3A illustrates another embodiment of a medicament seed 310 configured for enhanced retention within the gingival crevice in accordance with embodiments of the present disclosure.

FIG. 3B illustrates the medicament seed 310 after deposition into a gingival crevice in accordance with embodiments of the present disclosure.

FIG. 4A illustrates yet another embodiment of a medicament seed 410 configured for enhanced retention within the gingival crevice in accordance with embodiments of the present disclosure.

FIG. 4B illustrates the medicament seed 410 after deposition into a gingival crevice in accordance with embodiments of the present disclosure.

FIGS. 5A and 5B show an embodiment of a medicament seed 500 configured for enhanced retention within the gingival crevice in accordance with embodiments of the present disclosure.

FIGS. 6A and 6B illustrate the medicament seed 500 in its expanded state in accordance with embodiments of the present disclosure.

FIGS. 7A-7C illustrate an exemplary operational sequence for the deposition and retention of the medicament seed 500 within a patient's gingival crevice in accordance with embodiments of the present disclosure.

FIGS. 8A and 8B illustrate further embodiments of medicament seeds configured for enhanced retention within the gingival crevice in accordance with embodiments of the present disclosure.

FIG. 9A shows another embodiment of a medicament seed 900 configured for enhanced retention within the gingival crevice in accordance with embodiments of the present disclosure.

FIG. 9B shows another embodiment of a medicament seed 950 configured for enhanced retention within the gingival crevice in accordance with embodiments of the present disclosure.

FIG. 10 shows a high-level flow diagram 1000 of operations for a medicament seed configured with enhanced retention within the gingival crevice of a patient in accordance with embodiments of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses, or which render other details difficult to perceive may have been omitted. It should be understood, of course, that this disclosure is not limited to the particular embodiments illustrated herein.

### DETAILED DESCRIPTION

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features de-scribed herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. The Examiner, after having obtained a thorough understanding of the disclosure and claims of the present application has searched the prior art as disclosed in patents and other published documents, i.e., nonpatent literature. Therefore, the issuance of this patent is evidence that: the elements and limitations presented in the claims are enabled by the specification and drawings, the issued claims are directed toward patent-eligible subject matter, and the prior art fails to disclose or teach the claims as a whole, such that the issued claims of this patent are patentable under the applicable laws and rules of this country.

Various embodiments of the present disclosure are directed to enhance retention of medicaments deposited into the gingival crevice. For example, in order to facilitate direct and efficient medicament delivery to a patient's system, the present disclosure focuses on applications involving the gingival crevice as a primary route of administration. It is noted, however, that although the present disclosure is focused generally on enhancing retention of a medicament seed within the gingival crevice, it should be understood that in embodiments, the medicament seed may be configured to be retained, at least in part, in particular within the area of the gingival crevice adjacent or proximate the junctional epithelium portion of the gingival crevice.

It is noted that the oral cavity contains multiple gingival crevices, each providing an advantageous site for depositing medicament seeds that can dissolve and release their medicaments. By targeting these gingival crevices, and particularly the junctional epithelium located deep within them, the present disclosure leverages the high permeability of this anatomical region and its close proximity to connective tissue populated by immune cells. This approach not only improves bioavailability but also has the potential to reduce systemic side effects commonly associated with oral or injectable medicament delivery.

The inventors have found that the field of medicament delivery has seen a variety of routes, methods and devices developed over the years. The inventors have also found that these methods and devices may be employed to deliver a range of medicaments, including but not limited to nutrients, micronutrients, antibiotics, antigens, anti-inflammatory agents, antimicrobial agents, antibodies, steroids, DNA, and minerals. The delivery of these medicaments may be targeted to specific areas of the body to maximize their efficacy and minimize potential side effects. Additionally, the route of medicament delivery can have a significant impact on its pharmacokinetics and pharmacodynamics.

The route of delivery and the characteristics of the medicament itself, in its various forms may be factors that determine the amount that can be delivered, bioavailability, biodistribution, and the duration of circulation within the body. These factors are important in determining the choice of route of delivery for a given medicament. However, other concerns such as safety, efficacy, ease of administration, whether self-administered or given by a health care professional, costs, ease of manufacturing and stability of the medicament in the form determined by the route, also play significant roles. Matching the individual characteristics of the medicament to be delivered, in its various forms, to the routes through which it can be delivered, to accomplish the highest safety, efficacy, ease of administration, reduction of costs, and ease of manufacturing is a significant goal in developing novel medicament delivery systems. A novel device and route to deliver medicament to a space that has not been previously used for medicament delivery, the Gingival Crevice (GC), able to overcome many of the limitations of the commonly known routes of delivery, is described herein.

The classification of routes for administration of medicament is usually determined by the location of application. The two most commons routes of medicament delivery are broadly categorized as enteral (through the gastrointestinal tract), and parenteral (not enteral). The enteral route involves absorption of the medicament through the gastrointestinal (GI) tract. Administration can be through ingestion or other access to the GI tract such as tubes or catheters or insertion into the rectum. The parenteral route refers to any route that is not enteral. Parenteral administration can involve injection of medicament via a needle and syringe, or by insertion of an indwelling catheter into a body cavity, or by application of medicament on a surface of the body. Parenteral routes are often described with prefixes such as epi-(epidural), sub-(subcutaneous), intra-(intramuscular), extra-(extra-amniotic), peri-(perivascular) and trans-(transdermal or transmucosal) depending on where the medicament is deposited and how it is being taken up by the body.

The inventors have found that the enteral route is advantageous in that it is available for most individuals, is convenient, self-administrable, allows for systemic distribution and is often cost-effective. The disadvantages of the enteral route include the higher dosage of medicament required due to the barriers inherent to the GI tract. These barriers include, but are not limited to, the degradative acidic environment of the stomach, the first-pass effect (reductive filtration, enzymatic, and proteolytic process enacted through the liver during digestive processes resulting in significant removal of administered drug and thus reduced bioavailability for systemic circulation), and interactions with other medicaments. These disadvantages are magnified in situations where absorption has been surgically altered at the same time requirements for micronutrients are increased such as in the bariatric surgical population, or in situations where the enteral route is not available.

The inventors have found that the parenteral route is advantageous in that it can be rapid, can be delivered directly into the blood stream, can avoid the first-pass effect, can deliver much higher amounts of medicaments, can be utilized when the enteral route is not available and can be easily applied topically. The disadvantages of the parenteral route are that it can be painful, inconvenient, expensive, locally irritating, relatively difficult to store in some forms and may require administration by a health care professional. Both the enteral and parenteral routes of medicament delivery have limitations.

The GC, also known as the Gingival Sulcus, is a unique space that has special characteristics dictated by the environment it is in. The nature and the location of the GC requires it to have unique physical and biological characteristics as it is the barrier to the external environment and is under constant assault requiring it to be highly immunologically active. To facilitate this immune surveillance, the specialized mucosa of the GC is highly permeable allowing for rapid cellular motility and transport. The inventors have found that these unique characteristics make the GC an ideal target for immunomodulation. In addition, the high permeability of the GC also makes it an ideal site for medicament delivery, overcoming some of the limitations of both the enteral and commonly known parenteral routes. A novel device to deliver medicament to the GC, such as the devices described herein, can take advantage of these unique characteristics.

The GC is a dynamic fluid filled moat which is the interface between a tooth and the surrounding gingival tissue. It is a potential space filled with food debris, endogenous and exogenous cells, and chemicals. Any individual that has flossed is familiar with the characteristics of this potential space. In the deepest recesses of the GC is the Junctional Epithelium (JE) tissue which is a stratified squamous epithelium that resembles the oral epithelium but differs in some important ways. It is the only discontinuous epithelium layer in the body. The JE is the final barrier between what is inside the body and what is outside. Unlike the rest of the oral mucosa, cells of the JE are non-keratinized, have a high turnover rate, and have wide intercellular spaces which gives the JE its characteristic high permeability. The inventors have found that this high permeability of the JE makes it susceptible as a pathway for metabolic products of plaque bacteria, however this high permeability also makes it an ideal site for rapid absorption of medicament that are delivered to the GC. As the GC is the interface between the inside of the body and the outside world it is a highly immunological space. The JE expresses several cytokines and chemokines and is penetrated by neutrophil and Langerhans cells (specialized dendritic cells). The JE plays a key role in the innate immune system and in the overall health of gingival mucosa. The JE located at the interface between the gums and tooth surface, is a mucosal site. Additionally, the underlying connective tissue of the JE is vascularized. The vascularization of the underlying tissue helps to support immune responses and tissue repair in the gingiva. This vascularization enhances the JE's immune response by allowing immune cells to be recruited quickly during infection. This biological nature of the JE is crucial for maintaining its barrier function, immune defense, and periodontal health. The inventors have found that, as an active mucosal site and providing access to blood vessels in the underlying connective tissue, the JE can provide an advantage of inducing both mucosal and systemic responses, which makes the GC, specifically the JE of the GC, an ideal space for immunomodulation.

There are currently no methods for accessing the GC to deliver medicament to the deepest recesses to exploit the immune and absorptive potential of the junctional epithelium in the gingival crevice. A novel device designed to accurately target the deep GC, such as the devices and methods described herein, may take advantage of the unique characteristics of this space to safely, efficiently, consistently, quantifiably, painlessly and effectively deliver medicament through a novel route for systemic circulation and for immunomodulation.

This novel route of medicament delivery has not been previously described. It would fall under the general category of parenteral route of delivery. Specifically, such a route for medicament delivery may be characterized as a transmucosal route of delivery (diffusion through a mucosal membrane). Examples of transmucosal routes include insufflation (snorting of cocaine), sublingual (under the tongue) and sublabial (between the lips and gingiva), buccal (inner cheeks), and rectal (mucosal absorption but considered enteral as it is absorbed through the gastrointestinal tract). The JE of the gingival crevice, however, deserves special attention in that it is being utilized, in the present disclosure, for medicament delivery in a novel previously undescribed manner with a previously undescribed device. Unlike other transmucosal routes of delivery, the medicament is not placed into an open space, it is being wedged into a potential space that requires a novel device to deliver it. This potential space is the gingival crevice. The Merriam-Webster dictionary defines crevicular as: “of, relating to, or involving a crevice and especially the gingival crevice.” As such, the inventors have coined the phrase “Intra-crevicular route”. The route of delivery is thus referred to as the “INTRACREVICULAR ROUTE” of delivery. As the Junctional Epithelium is also unique and is the only epithelium in the body that is discontinuous, the route can also be referred to as the “TRANS-JUNCTIONAL ROUTE” and the delivery of medicaments can be appropriately referred to as “TRANS-JUNCTIONAL DELIVERY”.

The proposed novel route of junctional epithelium of the gingival crevice is unique and can be utilized to deliver an array of medicaments each with its unique therapeutic utility. The scope of some of the medicaments, their current challenges in terms of achieving therapeutic effect, and the potential of the JE as an alternative novel route for their delivery are discussed herein. Vaccines play a crucial role in protecting public health by preventing the spread of infectious diseases. They stimulate the immune system to recognize and fight specific pathogens, without causing the disease itself. This not only helps individuals build immunity but also reduces the overall prevalence of diseases, contributing to herd immunity. Vaccines have led to the eradication or significant reduction of deadly diseases like smallpox and polio. They are essential for controlling outbreaks, reducing healthcare costs, and improving life expectancy globally. Conventional vaccine delivery methods, including intramuscular (IM), subcutaneous (SC), and oral routes, have proven effective but come with significant limitations, especially in their ability to induce both systemic and mucosal immune responses. These routes are critical for preventing various infectious diseases, but each has its own set of challenges that affect their overall effectiveness, particularly for certain pathogens. IM and SC routes are commonly used for vaccines, especially for diseases requiring strong systemic immunity, such as hepatitis or tetanus. These methods ensure that the vaccine enters the bloodstream, where a systemic immune response can be triggered. However, the IM and SC routes fail to induce significant mucosal immunity, which is essential for defending against infections at incursion points like the respiratory or gastrointestinal tracts. Most viruses enter the human body through mucosal surfaces, such as the nose, mouth, eyes, and respiratory or gastrointestinal tracts. These sites serve as primary entry points for many pathogens thus it would be advantageous for a delivery route to allow for an induction of an antibody response at these mucosal sites in sync with systemic immune response, so that the attack from viruses can be thwarted at their point of entry. For diseases that primarily affect mucosal surfaces (e.g., influenza or enteric diseases), the conventional routes could be less effective because they might fail to engage the immune systems located in mucosal tissues. Vaccines that are delivered to the JE of the GC could overcome the limitations of conventional vaccine delivery routes and confer both systemic and mucosal immunity.

Micronutrient malnutrition affects over two billion individuals globally, with a particularly acute impact on children under five, more than half of whom suffer from vitamin and mineral deficiencies. Novel methods of micronutrient delivery, such as described herein, may help solve this problem. Micronutrients, which are not endogenously produced in the body-with the exception of vitamin D-must be obtained through dietary intake. In the United States alone, vitamin D deficiency is prevalent in 8% of the population, a figure that is likely much higher on a global scale. Essential minerals such as Iron, Zinc, and Iodine play a central role in preventing anemia, bolstering immune function, and supporting healthy cognitive development in infants. Similarly, Vitamin A is imperative for maintaining healthy eyesight and immune function, preventing blindness, and mitigating the risk of death from infections like measles and diarrhea.

Obese populations are particularly susceptible to micronutrient deficiencies due to factors such as fat sequestration, inflammation-associated malabsorption in the gut, and hyperinsulinemia-associated urinary excretion. These deficiencies are exacerbated with increasing obesity, especially among premenopausal obese females. The prevalence of morbid obesity and the corresponding rise in bariatric surgeries-recognized as the sole effective long-term weight loss treatment-further compound the issue. In 2022, the United States saw 280,000 bariatric procedures, reflecting a growing trend. Post-surgery, individuals experience a marked reduction in micronutrient absorption due to alterations in the stomach and small intestine, necessitating lifelong supplementation of oral vitamins and minerals to prevent deficiencies and associated diseases such as Wernicke's encephalopathy.

The conventional method of micronutrient supplementation for individuals who have undergone bariatric surgery typically involves the intake of a large number of pills, often exceeding twenty, multiple times throughout the day. This regimen, while effective in theory, presents a considerable burden to the individual. The sheer volume of pills and the frequency of administration can be overwhelming, leading to a decline in adherence over time. This lack of adherence can have serious health implications, as these individuals are already at a heightened risk of micronutrient deficiencies due to the nature of their surgical procedures.

Alternative methods of supplementation have been explored, but these too present their own set of challenges. Transdermal patches, for instance, have been introduced as a less invasive option. However, these patches have not demonstrated the same level of efficacy as oral supplementation. Clinical trials conducted by independent investigators have shown that these patches are considerably less effective than oral supplementation, resulting in higher rates of micronutrient deficiency when used as the sole method of supplementation.

Other forms of supplementation, such as subcutaneous or intramuscular injections, while effective, are not without their drawbacks. These methods can be quite costly, putting them out of reach for many individuals. Additionally, they can be painful, further discouraging adherence to the supplementation regimen.

Nasal sprays and sublingual applications have also been explored as alternative methods of supplementation. While these methods have been shown to be effective, they are limited in their scope. They are typically used for the delivery of specific vitamins, such as vitamin B12, and cannot provide a comprehensive solution for micronutrient supplementation. Furthermore, these methods can be quite expensive, further limiting their accessibility and use.

Other delivery methods, such as medicated floss, have proven to be less than ideal. The primary issue with these methods is that they fail to effectively deposit the micronutrients in the target area, which is the junctional epithelium of the gingival crevice. This is due to the fact that the medicament, or the substance carrying the micronutrients, is often scraped off outside of the gingival crevice during the process of flossing. As a result, a majority of the micronutrients do not reach the intended area and are instead wasted. This inefficiency in delivery not just reduces the effectiveness of the supplementation but also leads to wastage of the medicament. Moreover, these methods are often complex and require a high level of individual compliance for effectiveness. For instance, the use of medicated floss requires the individual to floss regularly and correctly, which can be a challenge for many. This complexity and the associated difficulty in maintaining regular use can lead to poor compliance, further reducing the effectiveness of these methods.

Pharmaceutical medications have greatly influenced health systems by enhancing treatment results and increasing life expectancy. They have changed the management of chronic conditions, including diabetes and hypertension, enabling people to lead healthier lives. Medications have transformed previously deadly conditions into more manageable ones, lowering death rates. Furthermore, the emergence of personalized medicine and targeted therapies has resulted in more efficient treatments, particularly in cancer treatment. Hormonal treatments have significantly changed lives, particularly for those with endocrine issues or experiencing life phases like menopause or hormone therapy post-surgery. For instance, thyroid hormone replacements assist in controlling hypothyroidism, whereas insulin therapy has been crucial for individuals with diabetes. Hormone replacement therapy (HRT) has reduced menopausal symptoms like hot flashes and mood changes, enhancing the quality of life for numerous women. These medications have enabled individuals to live healthier, more active lifestyles, offering relief from severe symptoms and facilitating improved control of chronic illnesses. In the end, pain medications and hormones have allowed individuals to take charge of their health, positively impacting their mental and physical wellness. Nonetheless, the method of drug delivery poses various challenges that can influence treatment efficacy and patient adherence. For instance, oral medications frequently encounter challenges with absorption and bioavailability, as they may be degraded in the digestive tract, diminishing their effectiveness. Injections, although more efficient for direct delivery, may lead to pain, discomfort, and local side effects such as swelling or irritation. Methods such as transdermal patches or sublingual tablets could enhance patient adherence by providing less frequent dosing, but they might not be appropriate for every kind of medication. Reaching controlled and precise drug release continues to pose a challenge, particularly with oral or transdermal techniques, and ensuring stability along with appropriate storage conditions complicates matters further. In addition, guaranteeing accurate drug administration to the designated location, such as in targeted therapies for cancer, remains a significant hurdle. These factors make choosing the right route of administration crucial for successful treatment.

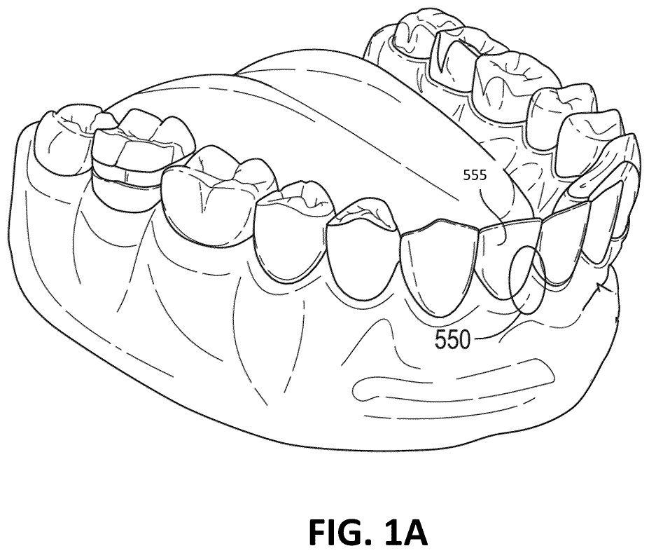

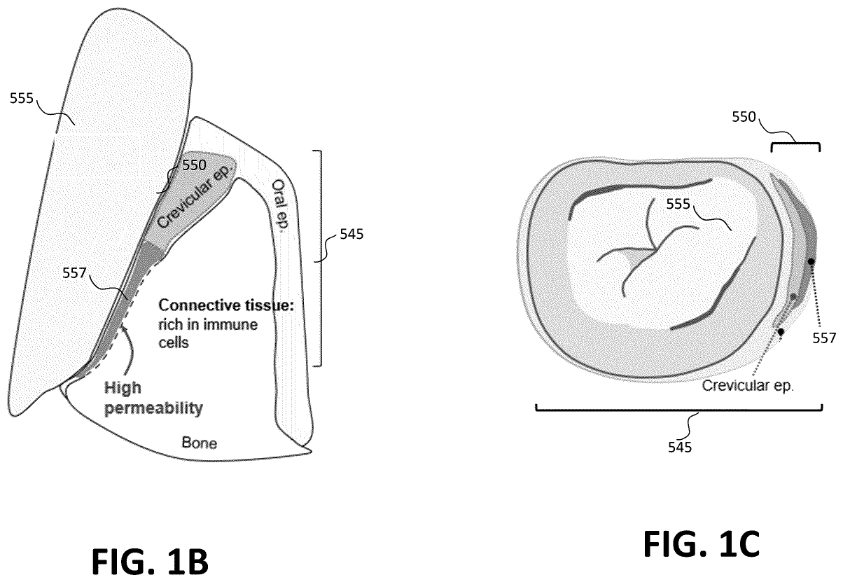

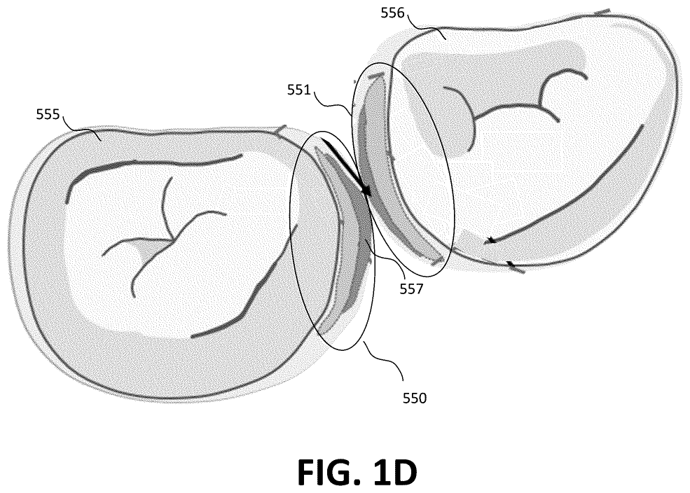

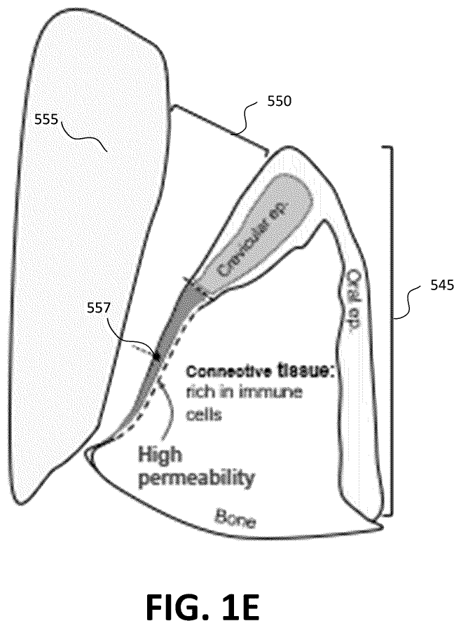

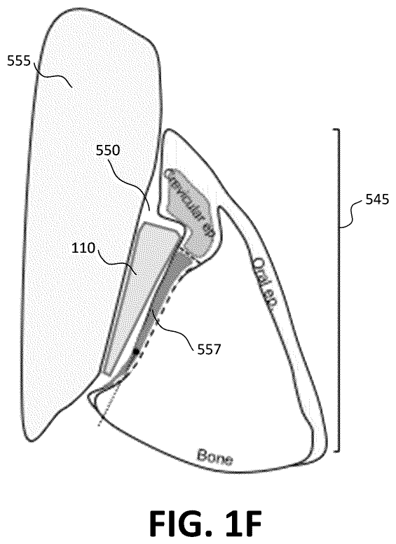

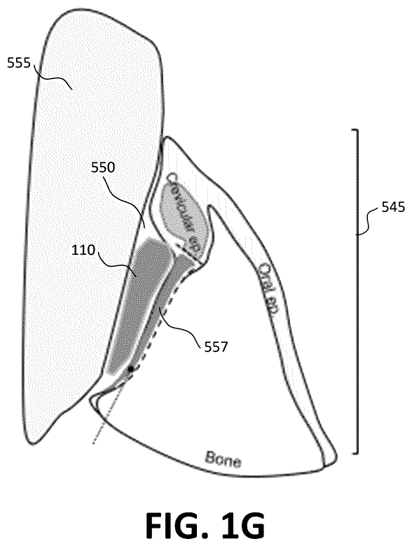

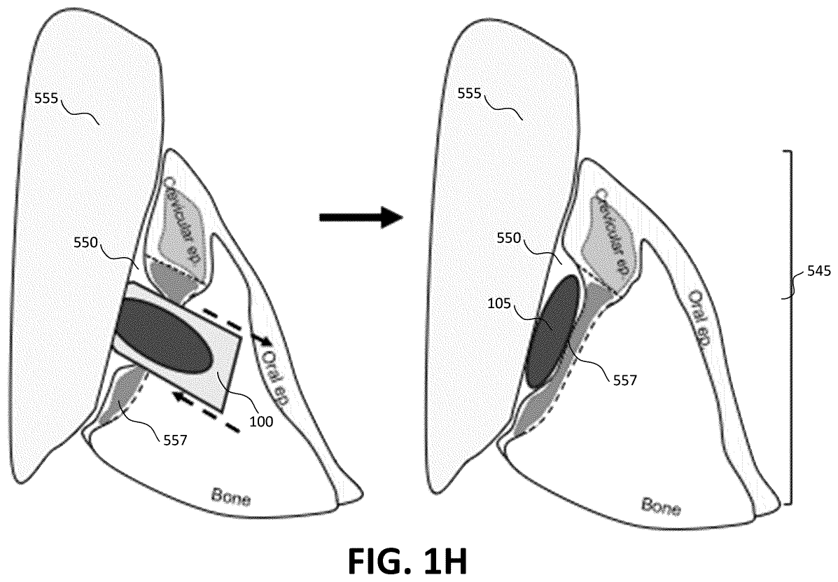

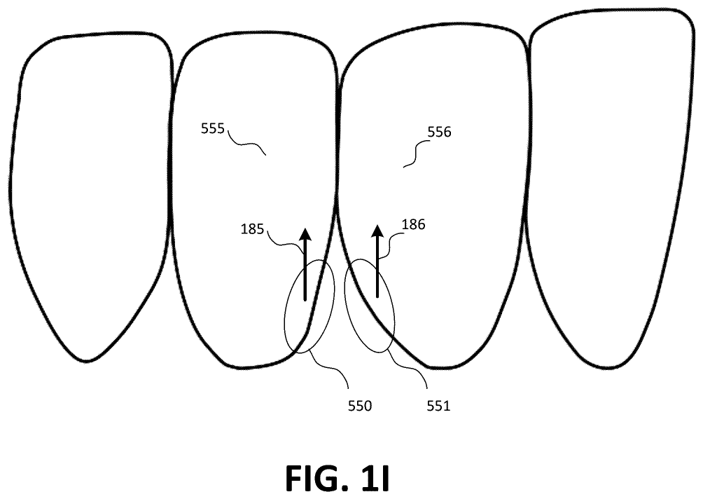

One approach is the delivery of medicaments through the gingival crevice 550, which may include the space between the tooth 555 and the tissue of the gingiva 545. FIG. 1A shows a perspective view of a portion of an oral cavity illustrating a representative gingival crevice 550 adjacent to a tooth 555 under normal conditions. Although FIG. 1A highlights a single tooth and its associated gingival crevice for clarity, it should be recognized that the oral cavity typically includes multiple gingival crevices. Each gingival crevice extends between the tooth surface and the gingival margin, continuing down to the junctional epithelium. Depositing a medicament seed within this space allows the medicament to be released gradually and absorbed rapidly through the specialized epithelial layer of the gingival crevice 550. The anatomical layout and physiological environment of the gingival crevice 550 will be discussed in more detail with reference to FIGS. 1B-1E.

More specifically, the junctional epithelium within the gingival crevice 550 shows considerable promise as a target for medicament delivery. The junctional epithelium has a high permeability due to its unique cellular structure and close contact with the underlying connective tissues, which are rich in immune cells. Because of this anatomical arrangement, the junctional epithelium allows medicaments to be rapidly absorbed directly into systemic circulation. As a result, this route has the potential to be more effective compared to conventional delivery methods.

FIGS. 1B-1E show an exemplary anatomy of a gingival crevice in the gingiva for targeting deposition and enhanced retention of medicament in accordance with embodiments of the present disclosure. It is noted that the illustration of the gingival crevice anatomy in FIGS. 1B-1E is intended to facilitate understanding the target region for medicament delivery as well as the challenges that arise from targeting the gingival crevice and the junctional epithelium for medicament delivery.

As shown, FIGS. 1B-1E illustrate detailed representations of the gingival crevice 550 within the gingiva 545, adjacent to the tooth 555. FIG. 1B shows a sagittal view of a representation of the tooth 555 and gingiva 545 under normal conditions. FIG. 1C shows a caudal (top-down) view that illustrates the overall geometry and orientation of the gingival crevice 550 relative to the tooth 555 structure. In embodiments, the gingival crevice 550 may be characterized as a potential space, which may indicate that although the crevicular epithelium and junctional epithelium 557 are normally in contact with the tooth 555, a space can be created within the gingival crevice 550 by insertion. This region contains two critical epithelial structures which may include the crevicular epithelium lining the crevice surface, which exhibits moderate bioabsorbability for medicament uptake, and the junctional epithelium 557 located deeper within the gingival crevice 550. The junctional epithelium 557 demonstrates high permeability, potentially attributable to its specialized cellular architecture and proximity to connective tissues rich in immune cells, allowing medicaments to permeate and be absorbed into the patient's system. In particular, the junctional epithelium 557 may include mucosa type tissue that may allow medicament deposited or released within the gingival crevice 550 to “seep through” the junctional epithelium 557 and to be absorbed into the patient's system. This anatomical arrangement may facilitate rapid, efficient absorption of therapeutic agents into the systemic circulation, which may provide and advantageous path for targeted medicament delivery.

FIG. 1D shows a top view of the exemplary anatomy of the gingival crevice 550 in the gingiva for targeting deposition of medicament in accordance with embodiments of the present disclosure. In particular, FIG. 1D shows two adjacent teeth, 555 and 556, and illustrates the anatomical layout of their corresponding gingival crevices 550 and 551. In this FIG. 1D, tooth 555 is associated with gingival crevice 550, while tooth 556 corresponds to gingival crevice 551. FIG. 1D illustrates the spatial relationship between the two gingival crevices 550 and 551, providing a depiction of how the gingival tissues interface with the tooth structures. In addition, this top-down view not only illustrates the positioning of the teeth 555 and 556 but also highlights the distinct anatomical characteristics of each gingival crevice 550 and 551. As shown, the gingival crevices 555 and 556 may be located at the juncture where the gingival margin meets the tooth surface, defining a narrow space that can be leveraged for the targeted deposition of medicament seeds for absorption through the junctional epithelium.

The inventors have found that achieving effective medicament delivery through the junctional epithelium 557 requires more than simply introducing a medicament into the general vicinity of the gingival crevice550. For example, merely applying a liquid or fluid formulation into the gingival crevice 550 may be insufficient for facilitating absorption through the junctional epithelium 557. This is partly because of the mucosal nature of the junctional epithelium itself, which means that effective permeation and subsequent systemic uptake may require a certain duration of contact time for the medicament to diffuse across this tissue barrier of the junctional epithelium 557. Fluids, lacking structure, may typically clear quickly from the gingival crevice 550 by natural processes like salivary flow and tissue movement, preventing sustained contact.

In addition, the physical nature of the gingival crevice 550 may present a challenge for fluid-based delivery. For example, the gingival crevice 550 is a potential space, which may mean that the opposing tissues (e.g., tooth surface and gingival epithelium) are normally in contact or close apposition. Creating and maintaining an actual space for medicament placement requires physical displacement or wedging of the tissues. A liquid may lack the physical structure to create the space. For example, a fluid cannot effectively wedge open the potential space upon application, or even prop the space open against the natural tendency of the tissues to return to contact.

In embodiments, the medicament seeds described in various embodiments of the present disclosure may be specifically configured to overcome these limitations. For example, the medicament seeds of embodiments may possess a sufficiently defined physical form (e.g., solid, semi-solid, semi-amorphous) having the structural integrity to interact mechanically with the gingival crevice 550's environment. Upon insertion, the medicament seed (or the deposition device arm in some embodiments) may operate as a wedge, physically creating a space between the tooth and the gingival tissues for deposition of the medicament seed.

The physical presence of the medicament seed within the gingival crevice 550 may not only create the space but may also maintain it for a duration determined by the medicament seed's properties (e.g., dissolution rate, etc.). By propping open the gingival crevice 550, the medicament seed may ensure sustained contact between the dissolving carrier, the released medicament, and the junctional epithelium 557. This may allow the medicament to effectively permeate the junctional epithelium 557 for absorption into the patient's system.

FIG. 1E shows a perspective view of a portion of an oral cavity illustrating a representative gingival crevice 550 adjacent to a tooth 555 under an open condition. For example, FIG. 1E presents an illustrative, somewhat exaggerated representation of the gingival crevice 550 to emphasize the potential space that may be utilized for medicament delivery in accordance with embodiments of the present disclosure. This view represents a scenario where the gingiva 545 is retracted or pulled away from the tooth 555, revealing the space of the gingival crevice 550 between the surface of the tooth 555 and the adjacent epithelial tissues. This visualization is intended to help in understanding the target anatomical space for the deposition of medicament seeds in accordance with embodiments of the present disclosure.

As shown, the created space within the gingival crevice 550 exhibits a generally V-shaped configuration, narrowing towards its apical end. Within this space, distinct regions may be identified relative to the epithelial lining. The upper region of the gingival crevice 550 space may be proximate to the crevicular epithelium, while the lower region may extend deeper, adjacent to the junctional epithelium 557. The junctional epithelium 557, as previously noted, includes characteristics highly favorable for systemic absorption.

The depiction in FIG. 1E underscores the objective of targeted medicament delivery within the gingival crevice 550. In particular, embodiments of the present disclosure may aim to insert a medicament seed such that it is deposited, at least partially, within the lower region of the gingival crevice 550, in close proximity to the junctional epithelium 557. Positioning the medicament seed in this apical area may leverage the high permeability of the junctional epithelium 557. For example, as the medicament seed dissolves and releases its medicament payload, the medicament may be presented to the junctional epithelium 557 for absorption into the patient's systemic circulation.

FIGS. 1F-1H shows various examples of medicament seed insertion into the gingival crevice in accordance with embodiments of the present disclosure. FIG. 1F shows an exemplary operational scenario following the insertion of a medicament seed 110 into the gingival crevice 550 adjacent to tooth 555. In this example, the medicament seed 110 may include a solid and dissolvable physical structure. As shown, the deposition of the medicament seed 110 into the gingival crevice 550 may physically displace the gingival tissue of the gingiva 545 away from the tooth 555 surface, creating an actual space within the potential gingival crevice 550 in which the medicament seed 110 may reside. The creation and maintenance of the space may be due to the medicament seed 110's solid form, which may enable it to resist the natural tendency of the gingival tissue to remain in close apposition to the tooth 555.

As also shown, the gingival tissue of the gingiva 545 may inherently seek proximity to the tooth 555, and may conform or wrap around the contours of the solid medicament seed 110. In embodiments, this may result in the upper region of the gingival crevice 550 remaining substantially closed or in contact with the tooth 555, while the medicament seed 110 may occupy the space more significantly in the lower region, particularly proximate to the junctional epithelium 557.

In embodiments, the tendency of the gingival tissue of the gingiva 545 to press against the inserted medicament seed 110 may generates inherent pressure within the gingival crevice 550. This pressure, larger in the deeper V-shaped portion near the junctional epithelium 557, may contribute to forces that may tend to displace or expulse the medicament seed 110 from the crevice 550. It is noted that retention mechanisms to counteract this displacement effect are discussed herein. Notwithstanding this displacement tendency, the placement shown in FIG. 1F positions the dissolvable medicament seed 110 ideally for medicament release. For example, as the medicament seed 110 dissolves over time, the medicament seed 110 may release the medicament directly into the maintained space adjacent to the highly permeable junctional epithelium 557, which may lead to absorption into the patient's system through the junctional epithelium.

FIG. 1G illustrates another embodiment of the medicament seed 110 positioned within the gingival crevice 550 adjacent to tooth 555. In this example, the medicament seed 110 may be configured with semi-amorphous properties, such as resembling a gel, paste, or other formulation that may not be fully solid nor fully fluid. In this example, the composition of the medicament seed 110 may provide sufficient structural integrity to physically create and maintain a space within the gingival crevice 550 upon deposition.

In embodiments, the semi-amorphous medicament seed 110 shown in FIG. 1G may be able to conform, at least partially, to the contours of the gingival crevice 550. For example, the medicament seed 110 may have enough structure to wedge the potential space open, but flexible enough to adapt to the anatomical geometry of the gingival crevice 550, which may enable the medicament seed 110 to potentially increase the surface area of contact between the medicament seed 110 and the surrounding gingival tissues of the gingiva 545, including the junctional epithelium 557.

In embodiments, despite its ability to conform, the semi-amorphous medicament seed 110 may effectively maintain the space within the gingival crevice 550 to ensure that the released medicament remains localized near the target absorption site. For example, as the medicament seed 110 dissolves or degrades within the gingival crevice 550, the medicament payload of the medicament seed 110 may be released proximate to the junctional epithelium 557 to be absorbed into the patient's system through the junctional epithelium 557.

FIG. 1H illustrates another embodiment of a medicament seed 105 positioned within the gingival crevice 550 adjacent to tooth 555 using a deposition device 100. In this embodiment, the medicament seed 105 may be formulated as a coating applied to at least a portion of the deposition device 100. In this example, the deposition device 100 carrying the medicament seed coating 105 may be inserted into the gingival crevice 550. The insertion of the device 100 may operate to create a space within the gingival crevice 550 between the gingiva 545 and the tooth 555.

Once the deposition device 100 is positioned within the gingival crevice 550, particularly near the desired target region such as the junctional epithelium 557, the medicament seed 105, which may be a coating, may be transferred from the deposition device 100 into the space of the gingival crevice 550. In embodiments, the transfer may be facilitated by some actions, such as agitating the deposition device 100, moving the deposition device 100 back and forth within the gingival crevice 550, or employing another mechanism configured to release or dislodge the coating (e.g., the medicament seed 105) from the surface of the deposition device 100. FIG. 1H shows both the deposition device 100 within the crevice and the released medicament seed 105 residing therein post-transfer.

In embodiments, following the release of the medicament seed 105 from the deposition device 100, the medicament seed 105, now present as deposited material within the gingival crevice 550, may dissolve and release the embedded or carried medicament into the gingival crevice 550 for absorption through the junctional epithelium 557 and into the patient's system.

As mentioned above, a primary objective for effective therapy is ensuring the medicament seed remains within the gingival crevice for a sufficient duration to permit dissolution and release of its medicament payload, allowing absorption through the junctional epithelium. However, achieving adequate retention within the gingival crevice presents significant challenges inherent to the anatomical configuration of the gingival crevice. For example, the anatomical configuration of the gingival crevice 550, particularly its V-shape which narrows towards the apical region adjacent to the junctional epithelium 557, naturally predisposes any deposited object, such as a medicament seed, to displacement forces directed upwards toward the broader opening of the gingival crevice. This tendency towards displacement may be further compounded by the dynamic oral environment, including the continuous flow of saliva and gingival crevice fluid, and movements of adjacent tissues, which may contribute to the premature migration of the medicament seed out of the gingival crevice 550 before it has fully dissolved and released its therapeutic payload.

FIG. 1I illustrates a front view of the gum pocket anatomy of a patient that may be targeted for medicament deposition. In particular, FIG. 1E shows a front view of teeth 555 and 556 along with their respective gingival crevices 550 and 551, illustrating their spatial relationship and the target area for medicament delivery. FIG. 1E illustrates the alignment of the teeth 555 and 556 and the adjacent gingival tissues, showing the positioning of the gingival crevices 550 and 551 where medicament seeds are targeted for deposition. The front view shows the relative placement of the gingival crevices 550 and 551 with respect to one another, providing an overall context of the oral anatomical environment and showing specific areas that may serve as targets for localized medicament delivery in accordance with embodiments of the present disclosure. It is noted that, a medicament seed deposited into one or more of the gingival crevices 550 and 551 may have a natural tendency to be displaced upwards toward the broader, upper region of the gingival crevice. For example, a medicament seed deposited into the gingival crevice 550 may have a natural tendency to be displaced upwards in direction 185 and a medicament seed deposited into the gingival crevice 551 may have a natural tendency to be displaced upwards in direction 186, which may cause the medicament seeds to migrate out of the respective gingival crevice before the medicament may be released.

What follows details various embodiments of the medicament seed configured for enhanced retention within the gingival crevice. In these embodiments, the medicament seed may operate as a carrier for therapeutic agents, such as micronutrient supplements, medicines, vaccines, etc., which may be intended for controlled release directly into the gingival crevice. By depositing the medicament seed into the targeted gingival crevice, the medicament seed may release its medicament payload gradually, ensuring that the therapeutic agent is efficiently absorbed through the permeable tissues of the crevicular and junctional epithelia.

In some embodiments, the medicament seed may be configured to provide a sustained and controllable release of the medicament. This controlled release mechanism may facilitate optimal bioavailability, as it may allow for the medicament to be absorbed over a predetermined period, reducing the need for frequent re-administration and enhancing the overall therapeutic efficacy. By focusing on the gingival crevice as the site of delivery (and in some embodiments in particular on the junctional epithelium), the disclosed system bypasses the challenges associated with other routes, such as degradation in the gastrointestinal tract, ensuring that a higher concentration of the active substance in the medicament reaches systemic circulation.

Nonetheless, retaining the medicament seed within the gingival crevice poses significant challenges. As discussed elsewhere herein, the inherent V-shaped anatomy of the gingival crevice and its dynamic, moist environment may contribute to the preferential displacement of the medicament seed from its intended site of deposition. To address these challenges, the medicament seed of embodiments may be configured for enhanced retention to ensure that the medicament seed remains securely in place within the gingival crevice until the medicament is fully released into the gingival crevice. The following description includes description of various medicament seed configurations that address these challenges.

The medicament seed may be configured to carry and deliver medicaments to the gingival crevice of the patient. For example, the medicament seed may be configured to, upon deposition into the gingival crevice of the patient, release medicament in a controlled manner, ensuring that the medicament is absorbed into the patient's system through the gingival crevice efficiently and effectively.

In embodiments, the medicament seed may have a semi-elastic or solid structure. This semi-elastic or solid structure may provide stability to the medicament seed during handling and insertion, while allowing for controlled dissolution once placed in the gingival crevice environment. In embodiments, a semi-elastic nature of the medicament seed may allow for some deformation during insertion, which may facilitate easier placement into the gingival crevice. In some embodiments, a solid structure may provide a more defined shape and controlled release profile.

In embodiments, the medicament seed may be composed of various materials, including polymers. The polymers may be selected based on their biocompatibility, dissolution properties, ability to carry and release the desired medicament, etc. In some embodiments, the medicament seed may be made from biodegradable polymers that break down naturally in the body over time. Examples of such polymers may include polylactic acid (PLA), polyglycolic acid (PGA), or copolymers of these materials.

In some embodiments, the medicament seed may be composed of hydrophilic polymers that absorb moisture from the gingival crevice environment, which may lead to swelling and eventual dissolution of the medicament seed. This particular property may allow for a gradual release of the medicament as the polymer matrix breaks down. In other embodiments, the medicament seed may be made from hydrophobic polymers that dissolve more slowly, which may provide a longer-lasting release of the medicament.

In embodiments, the size of the medicament seed may be configured to facilitate effective delivery and placement within the gingival crevice, while avoiding discomfort to the patient. In particular embodiments, the medicament seed may have a size ranging from 50 to 2000 microns (e.g., in diameter, length, width, and/or height). In some particular embodiments, such as where the medicament seed has an elongated shape, such as oblong, rectangular, or cylindrical configurations, the length of the medicament seed may range from 1 to 20 mm. These dimensions may enable optimal positioning of the medicament seed within the gingival crevice while providing sufficient volume for carrying an effective amount of medicament. The specific size and shape of the medicament seed may be selected based on factors such as the target treatment area, the type and amount of medicament to be delivered, the individual patient's gingival crevice anatomy.

In some embodiments, the medicament seed may be multi-modal. For example, the medicament seed may be composed or made up of several smaller sub-seeds. In embodiments, each of the sub-seeds may be configured to carry different types or amounts of medicament. In some embodiments, the sub-seeds within the multi-modal medicament seed may vary in size, shape, and/or composition. In some embodiments, each sub-seed may be configured to dissolve at a different rate, allowing for a multi-stage release of medicaments over time. For example, one sub-seed may be formulated to dissolve rapidly, providing an initial burst of medication, while others may be designed for slower, sustained release.

In some embodiments, the multi-modal medicament seed may include sub-seeds with different physical properties. For example, some sub-seeds may be hydrophilic, readily absorbing moisture and dissolving quickly, while others may be hydrophobic, dissolving more slowly. In some embodiments, the sub-seeds may be arranged in layers, with each layer designed to dissolve sequentially. In other embodiments, the sub-seeds may be distributed randomly throughout the medicament seed, which may provide a more uniform release of different medicaments over time. It is noted that a medicament seed with a multi-modal structure may enable allow the incorporation of incompatible medicaments within a single medicament seed. By encapsulating different medicaments in separate sub-seeds, it may be possible to deliver combinations of medicaments that may otherwise be chemically incompatible if mixed directly.

In some embodiments, the multi-modal medicament seed may include sub-seeds with different functions. For example, some sub-seeds may be configured to enhance adhesion to the gingival crevice, while others may be formulated to modify the local environment to improve the efficacy of the delivered medicaments.

In some embodiments, the medicament seed may be coated with a palatable substance to enhance patient comfort and acceptance. This coating may mask any unpleasant taste or texture of the medicament, potentially improving patient compliance with the treatment. In embodiments the palatable coating may be designed to dissolve quickly in the oral environment, allowing the medicament seed to begin its therapeutic action promptly after deposition in the gingival crevice.

In embodiments, the medicament seed may be configured to contain, include, encapsulate, or otherwise carry the medicaments to be delivered to the patient. In embodiments, the medicament seed may be configured to begin its function of medicament delivery upon deposition into the gingival crevice. This release of the medicament into the gingival crevice by the dissolution of the medicament seed may range from immediate release to a sustained release spanning seconds, minutes, hours, or even days.

In embodiments, the medicament seed may be configured to contain medicament in encapsulated polymer (e.g., entrapped in nanoparticle spheres), which include nanoparticle spheres encapsulating or entrapping medicament therein. In some embodiments, free form medicament (e.g., unencapsulated within nanoparticle spheres) may be concurrently contained in the solvent containing nanoparticles.

In some embodiments, the medicament seed may be loaded with micronutrients that include, but are not limited to, vitamins such as retinoid and carotene (Vitamin A), B-complex vitamins like thiamin (Vitamin B1), riboflavin (Vitamin B2), niacin (Vitamin B3), pantothenic acid (Vitamin B5), pyridoxine (Vitamin B6), cobalamin (Vitamin B12), biotin, and folic acid (Vitamin B9), ascorbic acid (Vitamin C), vitamins D (including calciferol, calcifediol, cholecalciferol, and ergocalciferol) and E (alpha-tocopherol), and Vitamin K, including phylloquinone and menadione. The micronutrients may include essential minerals such as calcium, chloride, chromium, copper, fluoride, iodine, iron, magnesium, manganese, molybdenum, phosphorus, potassium, selenium, sodium, sulfur, and zinc, and/or antibiotics such as amikacin, betamethasone, clindamycin, clotrimazole, gentamicin, kanamycin, minocycline, oxytetracycline, penicillin, and tetracycline.

In some embodiments, the medicament seed may include anti-inflammatory agents, antimicrobials, antifungals, and antibodies to combat various pathogens. Steroids can be included for their potent anti-inflammatory effects, while antiparasitic, anti-amoebic, anti-helminthic, anti-protozoal, antinematode, anti-cestode, and anti-trematode agents can be used to treat specific parasitic infections.

In some embodiments, the medicament seed may include vaccine/s to combat various pathogens. In this manner, the medicament seed of embodiments may be used for immunomodulation to combat future infections (such as, without limitation, influenza, human papilloma virus, coronavirus, etc.) by enabling the delivery of controlled doses of the vaccine/s directly to the gingival crevice specifically targeting the junctional epithelium.

In embodiments, the medicament seed may include allergy-inducing antigens, specific allergy-inducing molecules, or any combination thereof, to act as allergenic tolerance therapies, or to act as vaccines. In this manner, the medicament seed of embodiments may be used to treat and modulate tolerance to food allergies, such as peanut or shellfish allergies, as well as vaccinate individuals, by enabling the delivery of controlled doses of the allergens directly to the gingival crevice.

In embodiments, the medicament seed may be dissolvable. In this manner, the medicament seed may be configured to degrade to molecular form over a period of time after being deposited into the gingival crevice. In some embodiments, the dissolution of the medicament seed may allow for controlled release of the medicament into the surrounding tissue. The rate of dissolution of the medicament seed may vary depending on factors such as the composition and structure of the medicament seed, the environmental conditions in the gingival crevice, the specific medicament being delivered, etc. In some embodiments, the medicament seed may be designed to dissolve completely, leaving no residue in the gingival crevice after the medicament has been fully released.

In some other embodiments, the medicament seed may be non-dissolvable and may remain within the gingival crevice after delivering the medicament. In these embodiments, the medicament seed may be configured to release the medicament over time while maintaining structural integrity. After the medicament has been fully delivered, the non-dissolvable medicament seed may need to be removed from the gingival crevice. This removal process may include a separate procedure or the use of specialized tools to extract the medicament seed from the gingival crevice.

In embodiments, the medicament seed may be composed, in whole or in part, of a dissolvable polymer that is selected for its biocompatibility and dissolution characteristics. A variety of polymers can be utilized for this purpose, including but not limited to hydroxy acids, lactic acid polymers, glycolic acid polymers, poly (hydroxyl acids), poly(lactic acid) (PLA), poly(glycolic acid) (PGA), poly(lactic-co-glycolic acid) (PLGA), and copolymers with PEG, polyanhydrides, poly(ortho) esters, polyesters, polyurethanes, poly(butyric acid), poly(valeric acid), poly(caprolactone), poly(hydroxyalkanoates), poly(lactide-co-caprolactone), blends and copolymers thereof, natural polymers, proteins, albumin, collagen, gelatin, prolamines, zein, polysaccharides, alginate, cellulose derivatives and polyhydroxyalkanoates, polyhydroxy butyrate blends, and copolymers thereof.

In additional or alternative embodiments, natural polymers such as proteins, albumin, collagen, gelatin, prolamines, zein, and polysaccharides like alginate, as well as cellulose derivatives and polyhydroxy alkanoates, polyhydroxy butyrate blends, and copolymers thereof and other biologics, can be employed to form the medicament seed.

In embodiments, the polymers used in the construction of the medicament seed may be processed using solvents such as dichloromethane, ethyl acetate, acetone, or a combination thereof, to achieve the desired form and dissolution rate. As such, the medicament seed's configuration incorporates a dissolvable polymer matrix that responds to the environment of the gingival crevice providing functionality of the medicament seed to deliver medicaments over a controllable period of time. This configuration ensures that the medicament is released at a predetermined rate, providing a consistent and targeted therapeutic effect directly within the gingival crevice.

In embodiments, the rate of dissolution of the medicament seed may be configured to specific therapeutic requirements by adjusting the composition and properties of the medicament seed. For example, the rate of dissolution of the medicament seed may be configured by varying the type of polymer used for constructing the medicament seed, the concentration of the polymer (e.g., the density) of the medicament seed, the molecular weight of the polymer used for the medicament seed, the chain functional groups contents in the medicament seed, etc. In embodiments, the rate of dissolution may be increased or decreased by varying the above features. In some embodiments, the medicament seed may be configured with a multi-layered structure, where each layer has different dissolution properties, which may allow for phasic release of the medicament.

In embodiments, the rate of dissolution of the medicament seed may be configured based on the characteristics of the medicament seed, which may include the composition, structure, and/or surface energy of the polymer used in its construction. For example, for a slow and steady release of the medicament, the polymer may be selected or treated to dissolve at a slower rate, which may slow down the rate of dissolution in the moist environment of the gingival crevice. Conversely, for a more rapid release, the polymer may be treated or composed to dissolve at a faster rate subsequently increasing the rate of release of the medicament.

In embodiments, the medicament seed may be configured to carry the medicament using surface coating, in which at least a portion of the surface of the medicament seed may be coated with the medicament. In some embodiments, the medicament may be uniformly coated across the at least a portion of the surface of the medicament seed. In some embodiments, multiple coatings may be deposited over the surface of the medicament seed.

In embodiments, prior to coating with the medicament, a precoat layer may be applied to the medicament seed to modify the surface energy of the medicament seed, which may influence the dissolution rate and the subsequent release of the medicament. The precoat layer may also contain medicaments, which could be in free form, entrapped or encapsulated in particles (e.g., nanoparticles), encapsulated, or a combination thereof.

In embodiments, the medicaments with which the medicament seed may be coated may include medicaments in free form, entrapped or encapsulated in particles (e.g., nano-particles, encapsulated, or a combination thereof. In embodiments, the encapsulant for the medicament is selected from one or more polymers of the group consisting of poly(hydroxy acids), poly(lactic acid), Poly(glycolic acid), poly(lactic acid-co-glycolic acid), poly(lactic-co-glycolic acid), derivatives of poly(lactic-co-glycolic acid), PEGylated poly(lactic-co-glycolic acid), poly(lactide), poly(glycolide), poly(lactide-co-glycolide), poly(anhydrides), PEGylated poly(anhydrides), poly(ortho esters), derivatives of poly(ortho esters), PEGylated poly(ortho esters), poly(caprolactones), derivatives of poly(caprolactone), PEGylated poly(caprolactones), polyamines, spermine, spermidine, polylysine, and derivatives thereof, PEGylated polylysine, polyamides, polycarbonates, poly(propylene fumarates), polyamides, polyphosphazenes, polyamino acids, polyethers, polyacetals, polylactides, polyhydroxyalkanoates, polyglycolides, polyketals, polyesteramides, poly(dioxanones), polyhydroxybutyrates, polyhydroxyvalyrates, polycarbonates, polyorthocarbonates, poly(vinyl pyrrolidone), polycyanoacrylates, polyalkylene oxalates, polyalkylene succinates, poly(malic acid), poly(methyl vinyl ether), poly(ethylene imine), poly(acrylic acid), poly(maleic anhydride), poly(ethylene imine), derivatives of poly(ethylene imine), PEGylated poly(ethylene imine), poly (acrylic acid), derivatives of poly(acrylic acid), PEGylated poly(acrylic acid), poly(urethane), PEGylated polyurethane), derivatives of poly(urethane), poly (lactide), poly (glycolide), poly (hydroxyacids), polyesters, poly(arylates), polyalkylenes, polyethylene, polypropylene, polyalkylene glycols, poly(ethyleneglycol), polyalkylene oxides such as poly(ethylene oxide), polyalkylene terepthalates, poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinylacetate), polystyrene, polyurethanes and co-polymers thereof derivatized celluloses, alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutylmethyl cellulose, cellulose acetate, cellulose propionate, cellulose acetate butyrate, cellulose acetate phthalate, carboxylethyl cellulose, cellulose triacetate, cellulose sulfate sodium salt, polymers of acrylic acid, methacrylic acid or copolymers or derivatives thereof, methacrylic acid esters, poly(methyl methacrylate), poly(ethyl methacrylate), poly(butylmethacrylate), polyisobutyl methacrylate), poly(hexylmethacrylate), poly(isodecyl methacrylate), poly(laurylmethacrylate), poly(phenyl methacrylate), poly(methylacrylate), poly(isopropyl acrylate), poly(isobutyl acrylate), and poly(octadecyl acrylate), poly(butyric acid), poly(valeric acid), and poly(lactide-co caprolactone) and/or derivatives thereof, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly (methacrylic acid anhydride), methyl methacrylate, polymethacrylate, poly(methylmethacrylate) copolymer, polyacrylamide, aminoalkyl methacrylate copolymer, glycidyl methacrylate copolymers, polycyanoacrylates, and/or combinations thereof, natural polymers, proteins (such as albumin, collagen, gelatin), prolamines (for example, zein), polysaccharides (such as alginate), cellulose derivatives (such as hydroxypropyl cellulose, sodium carboxymethyl cellulose, ethyl cellulose and cellulose acetate), polyhydroxyalkanoates (for example, poly(hydroxy butyrate), and/or combinations thereof.

In embodiments, the medicament seed may be configured to carry the medicament using a polymer/medicament mixture, which may include integrating the medicament directly into the polymer matrix to form a polymer/medicament mixture. The polymer/medicament mixture may be used to form or make the medicament seed. In this manner, the medicament is not merely coated on the surface of the medicament seed but is distributed throughout the polymer material, making it an intrinsic part of the medicament seed's composition.

In embodiments, the polymer used for the polymer/medicament mixture may be a biocompatible, biodegradable polymer that is well-tolerated within the oral environment. The selection of the polymer may be based on its dissolution properties, compatibility with the medicament, and the desired release profile. Polymers such as poly(lactic acid), poly(glycolic acid), and their copolymers, including poly(lactic-co-glycolic acid), may be used due to their ability to degrade into non-toxic byproducts.

In embodiments, the medicament seed may be configured to carry the medicament using an internal well. For example, in embodiments, an internal well may be formed within the interior of the medicament seed. The internal well may be configured to encapsulate or trap the medicament within, which may be in liquid form or another suitable state for controlled release. The medicament seed may be configured to degrade or dissolve at a predetermined rate once placed within the moist environment of the gingival crevice. As the medicament seed begins to dissolve, it eventually breaches the internal well, allowing the encapsulated medicament to escape and be released into the gingival crevice. This release mechanism is activated by the dissolution of the medicament seed, which may be calibrated to occur over a controllable period.

In embodiments, the medicament seed 150 may be configured to carry the medicament using internal thread-like structures (monofilament) or mesh. In some embodiments, each thread of the internal thread-like structure may carry a different medicament portion and/or a different type of medicament. In some embodiments, these thread-like structures may be incorporated into a housing configured to dissolve. In embodiments, the housing and the internal thread-like structures may form a medicament seed.

In some embodiments, the internal well of the medicament seed may be configured to hold multiple types of medicaments, allowing for a combination therapy approach. For example, in some embodiments, the internal well may include multiple sub-wells, each sub-well configured to encapsulate and/or trap a different type and/or dosage of medicament. In some embodiments, each of the sub-wells may be configured to breach at different dissolution times of the medicament seed, which may allow for a multi-stage delivery of the medicament dosages. In some embodiments, the multiple sub-wells may be laid out side-by-side, or may be laid out one within the other (e.g., a first sub-well may be encapsulated by a second sub-well, etc.).

In embodiments, the medicament seed may be configured to carry the medicament using a multi-stage delivery configuration. The multi-stage delivery configuration may represent a combination of the features of the polymer/medicament mixture configuration and the internal well configuration. In the multi-stage delivery configuration, the medicament seed may be composed of a polymer/medicament mixture, and additionally, an internal well may be incorporated to encapsulate additional medicament. This configuration allows for a sequential release of medicaments. For example, in a first stage of the multi-stage delivery configuration of the medicament seed, as the medicament seed begins to dissolve within the gingival crevice, the medicament embedded within the polymer/medicament mixture may be gradually released. A second stage of the multi-stage delivery configuration may be initiated once the dissolution process reaches the internal well of the medicament seed, breaching its integrity and allowing the encapsulated medicament to be released into the gingival crevice. This second stage can be configured to occur after a predetermined time interval following the initial release, providing a subsequent boost or a different therapeutic action as dictated by the treatment plan. The encapsulated medicament within the internal well may include the same type of medicament as in the polymer/medicament mixture for a sustained dosage or may include a different type of medicament to address additional aspects of the patient's condition.

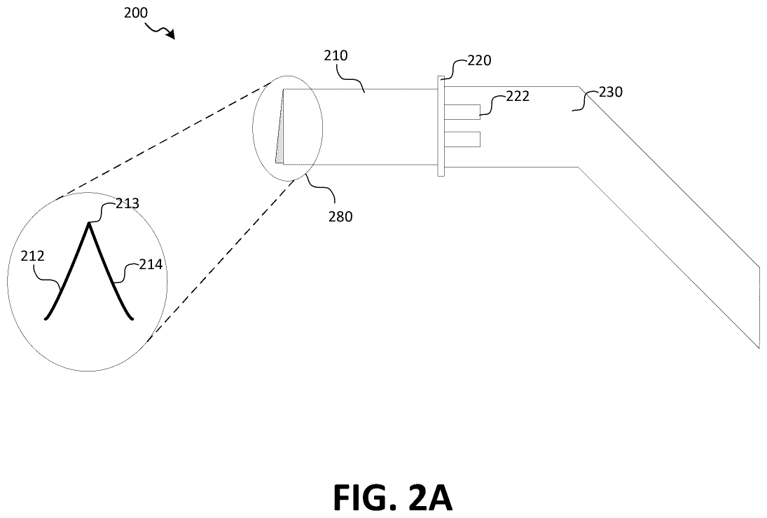

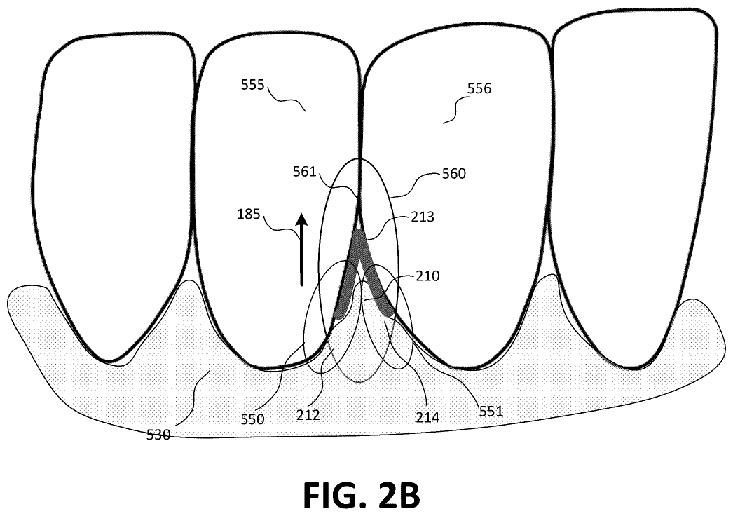

It is noted that any of the above described features and/or characteristics of the medicament seed may be used for any of the configurations of the medicament seeds described with respect to FIGS. 2A-9.

FIG. 2A illustrates medicament seed 210 configured for enhanced retention within the gingival crevice in accordance with embodiments of the present disclosure. In particular, the medicament seed 210 may be configured with a geometry and shape configured to enhance the retention of the medicament seed 210 within the gingival crevice. As shown in FIG. 2A, the medicament seed 210 may be configured to be used with a deposition device 200. The deposition device 200 may be configured to facilitate placement of the medicament seed 210 into the gingival crevice. The deposition device 200 may include several structural components, such as a handle 230, a backstop 220, and one or more tabs 222. In embodiments, the handle 230 may be configured to operate as the primary interface for manipulating and positioning the medicament seed 210 during the deposition procedure. By providing a secure grip and ergonomic design, the handle 230 may ensure that a user or a clinician can accurately guide the medicament seed 210 into the target site without undue force or the risk of slippage.

In embodiments, the backstop 220 may be positioned near the distal end of the deposition device 200. The backstop 220 may be configured to limit the depth to which the medicament seed 210 may be inserted into the gingival crevice. For example, the backstop 220 may be configured to operate as a physical barrier that prevents over-insertion of the medicament seed 210, helping to ensure that the seed is placed at the optimal depth within the gingival crevice.

In embodiments, the one or more tabs 222 may be positioned on or near the handle 230 and may be configured to engage or retain the medicament seed 210 to the handle 230 until the medicament seed 210 is correctly placed within the gingival crevice. Once the desired positioning of the medicament seed 210 is achieved, the tabs 222 may be configured to disengage, allowing the medicament seed 210 to separate from the deposition device 200 and remain securely in the gingival crevice. This separation mechanism may ensure that the medicament seed 210 stays in place without being inadvertently withdrawn when the deposition device 200 is removed.

As shown in FIG. 2A, the medicament seed 210 may be configured as an A-frame structure, shown in the exploded view 280. The A-frame configuration of the medicament seed 210 may include two legs, namely leg 212 and leg 214, that converge at a vertex 213. This geometric configuration may be configured to enhance retention of the medicament seed 210 within the gingival crevice by conforming to and/or leveraging the shape of the interdental papilla and adjacent gingival crevices of the target site. As discussed in greater detail below, each leg 212, 214 is intended to fit within and/or be inserted into a respective gingival crevice, while the vertex 213 may be configured to abut or wedge against the interdental papilla or dental junction, helping to prevent upward or lateral displacement of the medicament seed 210 once the medicament seed 210 is deposited.

FIG. 2B illustrates the medicament seed 210 after deposition into one or more gingival crevices in accordance with embodiments of the present disclosure. For example, during operation, the medicament seed 210 may be deposited into one or more gingival crevices using the deposition device 200. In particular, the deposition device 200 may be used to align the medicament seed 210 and to insert it into gingival crevices 550 and 551. In particular, the legs leg 212 and 214 of the medicament seed 210 may be inserted, respectively, into the gingival crevices 550 and 551. Once the medicament seed 210 is within the targeted deposition site, the medicament seed 210 may be released from the deposition device 200 and may be left within the gingival crevices 550 and 551. For example, a user may break off the medicament seed 210 from the deposition device 200 by twisting or otherwise causing the one or more tabs 222 to release the medicament seed 210 from the deposition device 200. In some embodiments, the user may trigger a mechanism that releases the medicament seed 210 from the deposition device 200, allowing the medicament seed 210 to separate and be left in place within the gingival crevices 550 and 551.

FIG. 2B shows the medicament seed 210 in its intended operational position within the oral cavity, having been deposited into gingival crevices 550 and 551. In this embodiment, teeth 555 and 556 may be adjacent to one another, meeting at a dental junction 561, while the interdental papilla 560 may be situated between them. As shown, each leg 212 and 214 of the A-frame configuration of the medicament seed 210 may be inserted into a respective gingival crevice, with the vertex 213 abutting the dental junction 561. In this position and orientation, the natural inverted V-shaped contour of the interdental papilla 560 is leveraged to help to secure the medicament seed 210 in place and reduce the risk of displacement.

In this manner, the vertex 213 of the medicament seed 210 effectively anchors the medicament seed 210 at the apex of the interdental papilla 560, ensuring that the legs 212 and 214 extend into the crevices 550 and 551, respectively, at an optimal depth and are retained therein. Because of this snug fit, the medicament seed 210 may be significantly less affected by the natural pressure exerted by the gingival crevice to expulse the medicament seed 210 therefrom. The stable positioning of the medicament seed 210 also ensures that the medicament seed 210 remains in continuous contact with the gingival tissue, and provides sufficient time for the medicament seed 210 to dissolve ands for the medicament to be absorbed into the patient's system via the junctional epithelium.

In embodiments, the A-frame structure of the medicament seed 210 not only wedges the medicament seed 210 against the interdental papilla 560 but also prevents it from being displaced upward (in direction 185). By anchoring the vertex 213 at the dental junction 561, the medicament seed 210 is effectively “locked” within the gingival crevices, minimizing any tendency to migrate out of the targeted area. This ensures the efficacy of the medicament seed 210, as it provides a controlled environment in which dissolution and absorption may take place without interruption.

In embodiments, the configuration of the A-frame may enable each leg 212 and 214 to conform to the unique anatomy of its respective gingival crevice. Even in patients where crevice depths or shapes may vary, the geometry of the medicament seed 210 may allow for a secure fit that adapts to these individual differences. As the medicament seed 210 gradually dissolves, it releases active ingredients that are absorbed into the patient's system through the junctional epithelium agent.

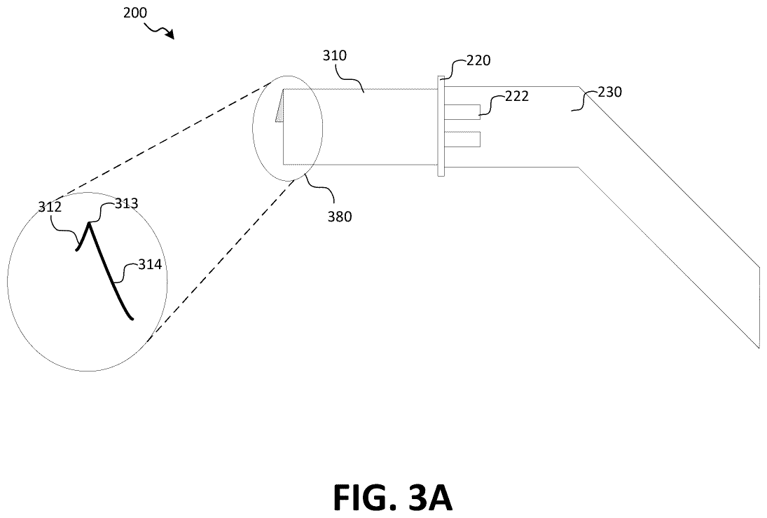

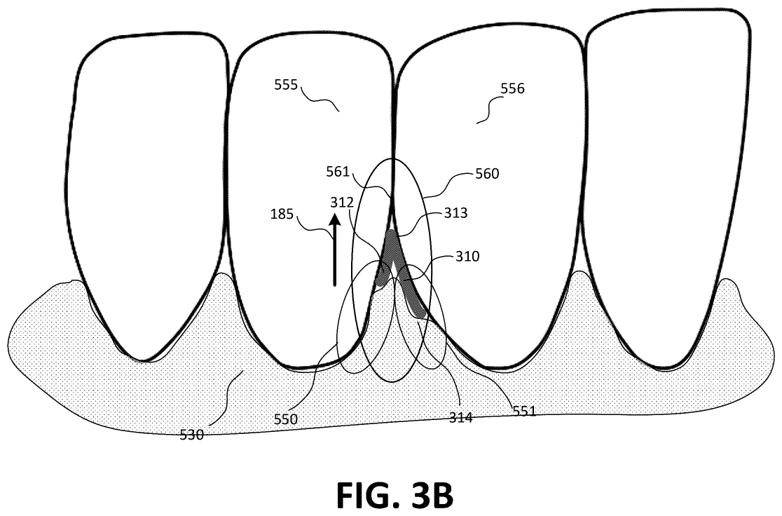

FIG. 3A illustrates another embodiment of a medicament seed 310 configured for enhanced retention within the gingival crevice in accordance with embodiments of the present disclosure. Similar to the embodiment shown in FIG. 2A, the medicament seed 310 may be configured with a geometry and shape configured to enhance the retention of the medicament seed 310 within the gingival crevice.

As shown in FIG. 3A, the medicament seed 310 may be configured to be used with a deposition device 200. The deposition device 200 may be configured to facilitate placement of the medicament seed 310 into the gingival crevice. The deposition device 200 may include several structural components, such as a handle 230, a backstop 220, and one or more tabs 222, which may include functionality and may operate similar to the description with respect to FIG. 2A.

As shown in FIG. 3A, the medicament seed 310 may be configured as a modified A-frame structure, shown in the exploded view 380. Similar to the A-frame configuration of the medicament seed 210 shown in FIG. 2A, the medicament seed 310 may include two legs, namely leg 312 and leg 314, that converge at a vertex 313. However, in this embodiment, leg 312 may be configured to be shorter than leg 314. This geometric configuration may be configured to enhance retention of the medicament seed 310 within the gingival crevice by conforming to and/or leveraging the shape of the interdental papilla and adjacent gingival crevices of the target site, similar to the embodiment of FIGS. 2A and 2B.

FIG. 3B illustrates the medicament seed 310 after deposition into a gingival crevice in accordance with embodiments of the present disclosure. For example, during operation, the medicament seed 310 may be deposited into one or more gingival crevices using the deposition device 200. In particular, the deposition device 200 may be used to align the medicament seed 310 and to insert it into gingival crevice 551. In this case, unlike the embodiment of FIGS. 2A and 2B where both legs 212 and 214 are inserted into respective gingival crevices 550 and 551, in the embodiment of FIG. 3B, only the longer leg 314 of the medicament seed 310 may be inserted into the gingival crevice 551, while the shorter leg 312 may not extend significantly into the gingival crevice 550, or may not extend into it at all.

In embodiments, this configuration shown in FIGS. 3A and 3B may offer several advantages because the placement does not need to precisely account for the specific geometry of the oral cavity of the patient, which may vary significantly between patients (e.g., variations in crevice depth or the size of the interdental papilla, among many differences). This allows for greater flexibility in the design and dimensioning of the leg 314 that is intended for insertion into the gingival crevice 551.

In embodiments, once the medicament seed 310 is within the targeted deposition site, the medicament seed 310 may be released from the deposition device 200 and may be left within the gingival crevices 550 and/or 551 or proximate thereto. For example, a user may break off the medicament seed 310 from the deposition device 200 by twisting or otherwise causing the one or more tabs 222 to release the medicament seed 310 from the deposition device 200. In some embodiments, the user may trigger a mechanism that releases the medicament seed 310 from the deposition device 200, allowing the medicament seed 310 to separate and be left in place.

FIG. 3B shows the medicament seed 310 in its intended operational position within the oral cavity, having been deposited proximate to gingival crevices 550 and/or 551. In this embodiment, teeth 555 and 556 may be adjacent to one another, meeting at a dental junction 561, while the interdental papilla 560 may be situated between them. As shown, the longer leg 314 of the modified A-frame configuration of the medicament seed 310 may be inserted into gingival crevice 551, while the vertex 313 abuts the dental junction 561. The shorter leg 312 rests proximate to the gingival crevice 550 and may or may not be inserted into gingival crevice 550. In this position and orientation, similar to the embodiment of FIGS. 2A and 2B, the natural inverted V-shaped contour of the interdental papilla 560 is leveraged to help secure the medicament seed 310 in place and reduce the risk of displacement.

In this manner, the vertex 313 of the medicament seed 310 effectively anchors the medicament seed 310 at the apex of the interdental papilla 560, ensuring that the longer leg 314 extends into the crevice 551 at an optimal depth and is retained therein, while the shorter leg 312 provides stability without requiring deep insertion into crevice 550. Because of this snug fit achieved by the wedging action of the vertex 313 against the interdental papilla 560, the medicament seed 310 may be significantly less affected by the natural pressure exerted by the gingival crevice to expulse the medicament seed 310 therefrom. This configuration still effectively causes the vertex 313 of the A-frame shaped medicament seed 310 to wedge against the interdental papilla 560, retaining it within or proximate to the gingival crevice 551 and preventing displacement. This simplified configuration also offers potential savings in materials compared to the embodiment with two equally long legs.

The stable positioning of the medicament seed 310 also ensures that the medicament seed 310 remains in continuous contact with the gingival tissue (particularly within crevice 551), and provides sufficient time for the medicament seed 310 to dissolve and for the medicament to be absorbed into the patient's system via the junctional epithelium.

In embodiments, the modified A-frame structure of the medicament seed 310, with one shorter leg 312, not only wedges the medicament seed 310 against the interdental papilla 560 but also prevents it from being displaced upward (in direction 185). By anchoring the vertex 313 at the dental junction 561, the medicament seed 310 is effectively “locked” proximate to the gingival crevices, minimizing any tendency to migrate out of the targeted area. This ensures the efficacy of the medicament seed 310, as it provides a controlled environment in which dissolution and absorption may take place without interruption.

In embodiments, the configuration of the modified A-frame may enable the longer leg 314 to conform to the unique anatomy of its respective gingival crevice 551. Even in patients where crevice depths or shapes may vary, the geometry of the medicament seed 310 may allow for a secure fit that adapts to these individual differences, particularly for the longer leg 314 which is primarily responsible for engaging the gingival crevice 551. As the medicament seed 310 gradually dissolves, it releases active ingredients that are absorbed into the patient's system through the junctional epithelium.

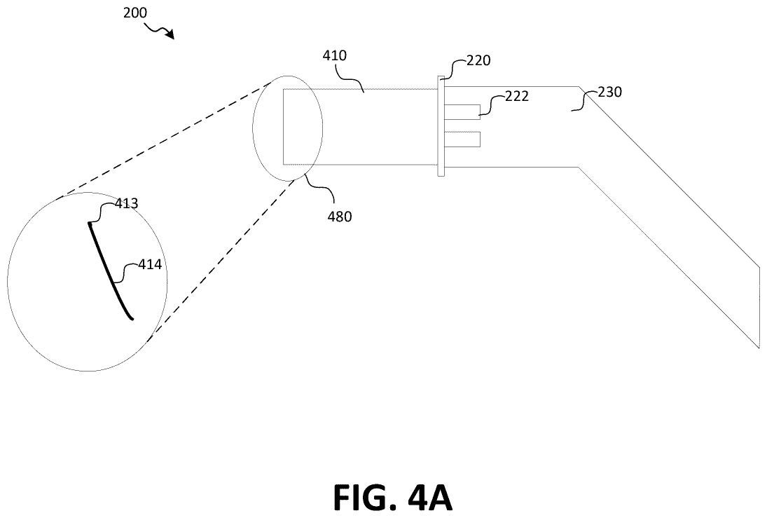

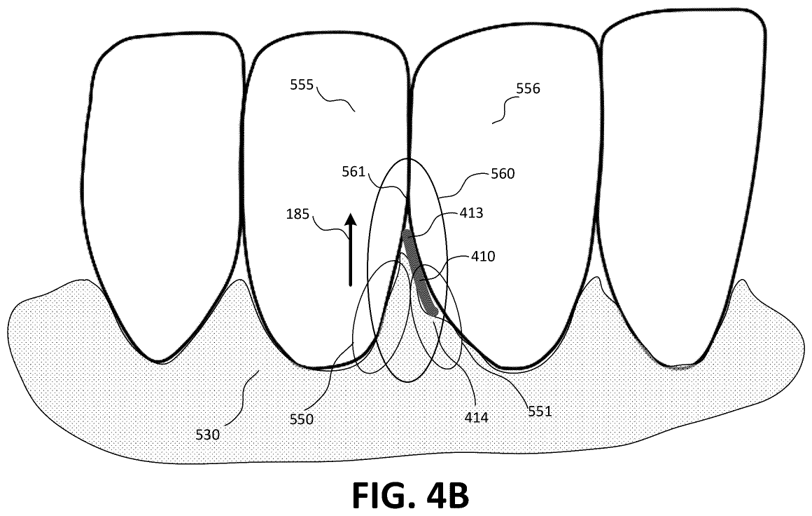

FIG. 4A illustrates yet another embodiment of a medicament seed 410 configured for enhanced retention within the gingival crevice in accordance with embodiments of the present disclosure. Further simplifying the designs shown in FIGS. 2A and 3A, the medicament seed 410 may be configured with a specific geometry and shape configured to enhance the retention of the medicament seed 410 within the gingival crevice.

As shown in FIG. 4A, the medicament seed 410 may be configured to be used with a deposition device 200. The deposition device 200 may be configured to facilitate placement of the medicament seed 410 into the gingival crevice. The deposition device 200 may include several structural components, such as a handle 230, a backstop 220, and one or more tabs 222, which may include functionality and may operate similar to the description with respect to FIG. 2A.

As shown in FIG. 4A, the medicament seed 410 may be configured as a single leg structure, shown in the exploded view 480. This represents a further simplification from the two-legged A-frame structures of medicament seed 210 (FIG. 2A) and medicament seed 310 (FIG. 3A). In embodiments, the single-leg configuration of the medicament seed 410 may include a body or leg 414 and an end or vertex 413. This geometric configuration may be configured to enhance retention of the medicament seed 410 within the gingival crevice by conforming to and/or leveraging the shape of the interdental papilla and an adjacent gingival crevice of the target site.

FIG. 4B illustrates the medicament seed 410 after deposition into a gingival crevice in accordance with embodiments of the present disclosure. For example, during operation, the medicament seed 410 may be deposited into a gingival crevice using the deposition device 200. In particular, the deposition device 200 may be used to align the medicament seed 410 and to insert it into a single gingival crevice, such as gingival crevice 551. The leg 414 of the medicament seed 410 may be inserted into the gingival crevice 551 such that the end or vertex 413 abuts or wedges against the dental junction 561 or the apex of the interdental papilla 560. This single-leg configuration significantly simplifies both the manufacturing and placement process.

Once the medicament seed 410 is within the targeted deposition site, the medicament seed 410 may be released from the deposition device 200 and may be left within the gingival crevice 551. For example, a user may break off the medicament seed 410 from the deposition device 200 by twisting or otherwise causing the one or more tabs 222 to release the medicament seed 410 from the deposition device 200. In some embodiments, the user may trigger a mechanism that releases the medicament seed 410 from the deposition device 200, allowing the medicament seed 410 to separate and be left in place within the gingival crevice 551.

FIG. 4B shows the medicament seed 410 in its intended operational position within the oral cavity, having been deposited into gingival crevice 551 adjacent to tooth 556. In this embodiment, teeth 555 and 556 may be adjacent to one another, meeting at a dental junction 561, while the interdental papilla 560 may be situated between them. As shown, the leg 414 of the medicament seed 410 may be inserted into the gingival crevice 551, with the end or vertex 413 abutting the dental junction 561. In this position and orientation, similar to the embodiments of FIGS. 2A-2B and 3A-3B, the natural inverted V-shaped contour of the interdental papilla 560, and particularly the apex of the dental junction 561, is leveraged to help secure the medicament seed 410 in place and reduce the risk of displacement.

In this manner, the end or vertex 413 of the medicament seed 410 effectively anchors the medicament seed 410 at or near the apex of the interdental papilla 560, ensuring that the leg 414 extends into the crevice 551 at an optimal depth and is retained therein. Because of this snug fit achieved by the wedging action of the vertex 413 against the interdental papilla 560 or dental junction 561, the medicament seed 410 may be significantly less affected by the natural pressure exerted by the gingival crevice to expulse the medicament seed 410 therefrom. This configuration still effectively causes the vertex 413 of the shaped medicament seed 410 to wedge against the interdental papilla 560, retaining it within the gingival crevice 551 and preventing displacement, but with a much simpler design that may offer further savings in materials and ease of manufacturing.

The stable positioning of the medicament seed 410 also ensures that the medicament seed 410 remains in continuous contact with the gingival tissue within crevice 551, and provides sufficient time for the medicament seed 410 to dissolve and for the medicament to be absorbed into the patient's system via the junctional epithelium.

In embodiments, the single-leg structure of the medicament seed 410 wedges the medicament seed 410 against the interdental papilla 560 and prevents it from being displaced upward (in direction 185). By anchoring the vertex 413 at the dental junction 561, the medicament seed 410 is effectively “locked” within the gingival crevice 551, minimizing any tendency to migrate out of the targeted area. This ensures the efficacy of the medicament seed 410, as it provides a controlled environment in which dissolution and absorption may take place without interruption.

In embodiments, the configuration of the single leg 414 may enable it to conform to the unique anatomy of its respective gingival crevice 551. Even in patients where crevice depths or shapes may vary, the geometry of the medicament seed 410 may allow for a secure fit that adapts to these individual differences. As the medicament seed 410 gradually dissolves, it releases active ingredients that are absorbed into the patient's system through the junctional epithelium.

A potential issue that may arise with using the A-frame (e.g., medicament seed 210, 310) or single-leg (e.g., medicament seed 410) medicament seed configurations described herein may be due to the natural anatomical variation observed among patients. Specifically, the dimensions of the interdental papilla (e.g., interdental papilla 560), including its height and width, may differ significantly from one individual to another. Some patients may have relatively long or large interdental papillae, while others may have comparatively short or smaller ones. This variation may present a challenge for using a universally sized medicament seed, as a medicament seed configured for an average papilla size may not achieve the desired wedging effect and secure retention in patients with substantially different papillary dimensions.

To address these challenges, a patient-specific approach may be implemented in some embodiments. This approach may include utilizing a sizing tool or device, such as a sizer, that may be configured to measure the dimensions of a specific patient's interdental papilla and associated gingival crevices. Based on the measurements obtained using the sizer, the optimal dimensions for the A-frame or single-leg medicament seed (e.g., including the length of the leg or legs, and vertex angle or shape), may be determined for that particular patient. Subsequently, a medicament seed precisely configured to the patient's unique anatomy may be manufactured or selected and provided to the patient or clinician for deposition. In this manner, the likelihood of ensuring secure wedging against the interdental papilla and effective retention within the gingival crevice may be maximized.

In embodiments, the medicament seed may be configured with a material consistency configured to promote and enhance retention of the medicament seed within the gingival crevice. This may include configuring the medicament seed to conform or otherwise adapt to the anatomical shape and contours of the patient's gingival crevice upon deposition therein. By conforming to the specific geometry of the gingival crevice, the medicament seed may achieve a more intimate contact with the surrounding tissues and may increase frictional forces and potentially bioadhesion, which may operate to resist displacement forces of the gingival crevice and maintain the medicament seed securely within the gingival crevice for the required duration to ensure dissolution and absorption of the medicament into the patient's system through the junctional epithelium.

For example, in some embodiments, the medicament seed may be formulated and configured as a gel and/or as a highly viscous fluid. When the gel and/or highly viscous fluid medicament seed is deposited into the gingival crevice, the material properties of the medicament seed may allow it to slowly flow and spread, gradually creating and/or filling the spaces within the gingival crevice. This flow characteristic may enable the medicament seed to conform to the shape of the patient's gingival crevice.

In some embodiments, using a viscous or gel-like formulation, the medicament seed may be configured to undergo a change in its physical state after deposition into the gingival crevice. For example, the viscous medicament seed may be configured to harden, either slightly or potentially fully, over a predetermined period following its placement within the gingival crevice environment. This hardening may transform the initially flowable medicament seed into a more solid or semi-solid structure that may be firmly molded to the contours of the gingival crevice. In this manner, the hardened medicament seed may remain securely lodged within the gingival crevice, resisting displacement forces until dissolution and absorption into the patient's system through the junctional epithelium.

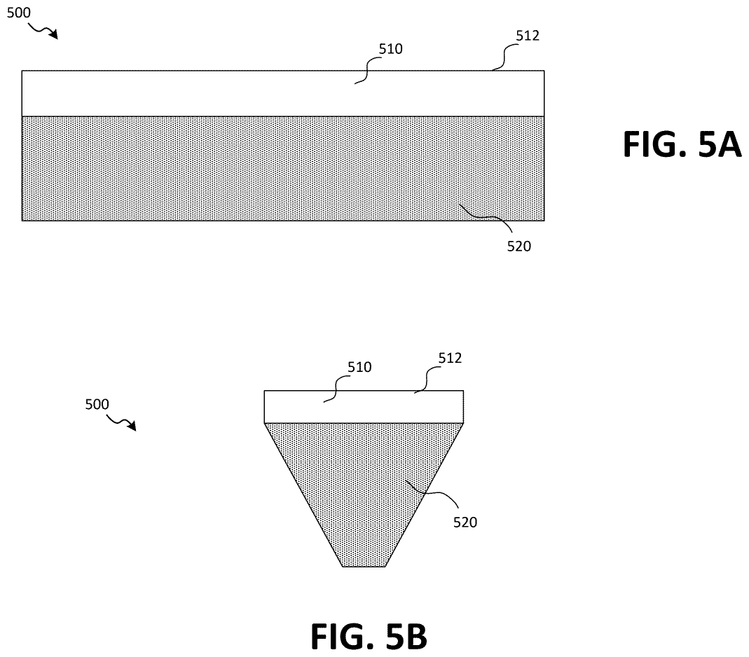

FIGS. 5A and 5B show an embodiment of a medicament seed 500 configured for enhanced retention within the gingival crevice in accordance with embodiments of the present disclosure. In embodiments, the medicament seed 500 may include a dynamically expanding component within the structure of the medicament seed 500 that may operate to enhance the retention of the medicament seed 500 within the gingival crevice. In particular, FIG. 5A shows a side view of the medicament seed 500 in its initial, pre-expansion or compressed state, representing the configuration prior to deployment or activation within the gingival crevice. FIG. 5B shows a frontal view of the medicament seed 500 in its pre-expansion or compressed state. As shown, the medicament seed 500 may include an expanding section 510, and a medicament section 520. In embodiments, the medicament section 520 may be configured to carry the medicaments, including micronutrients, pharmaceuticals, vaccines, or other therapeutic agents as described elsewhere herein, and may be configured to be dissolvable in accordance with the description herein.

The expanding section 510, shown in the compressed state may be retained in a compressed configuration relative to its potential expanded state. As shown, the expanding section 510 may include a restraining mechanism 512 configured to keep the expanding section 510 in its compressed state. In some embodiments, the expanding component 510 may be made from a material capable of significant volumetric expansion when unconstrained and exposed to moisture.

In embodiments, the restraining mechanism 512 may be made from a material selected to degrade or dissolve upon exposure to the conditions within the gingival crevice, particularly moisture from saliva or gingival crevicular fluid. For example, the restraining mechanism 512 may be a biocompatible, dissolvable polymer film or coating, membrane, etc. encapsulating the expanding section 510. The composition of the restraining mechanism 512 may be configured to control the time required for its dissolution or degradation, determining when the expanding component 510 may begin to expand after deposition within the gingival crevice.

In some embodiments, the restraining mechanism 512 may include a polymer holder or collar structure, which may be made from dissolvable materials configured to degrade, dissolve, or otherwise mechanically release the constraint on the expanding section 510 upon sufficient exposure to the environment of the gingival crevice. The characteristics of the dissolution or degradation of the restraining mechanism 512 may be configured to control the timing of the expansion of the expanding section 510.

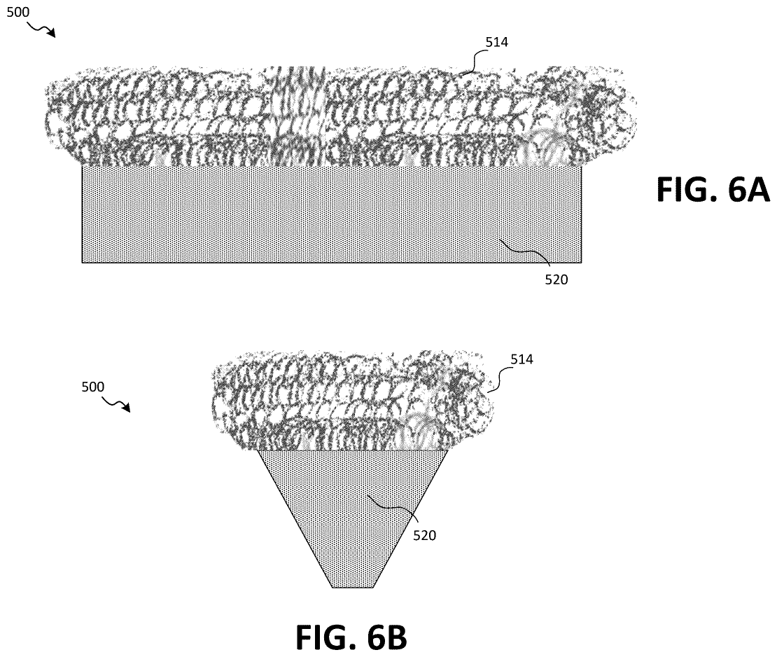

FIGS. 6A and 6B illustrate the medicament seed 500 in its expanded state in accordance with embodiments of the present disclosure. In embodiments, the expanded state of the medicament seed 500 may follow the release by the restraining mechanism 512. In particular, FIG. 6A shows a side view of the expanded medicament seed 500 and FIG. 6B shows a frontal view of the expanded medicament seed 500. As mentioned above, upon placement of the medicament seed 500 into the gingival crevice, the restraining mechanism 512 may be exposed to the environment of the gingival crevice, which may cause the restraining mechanism 512 to dissolve, degrade, or otherwise fail, releasing the constraint on the compressed expanding section 510 and causing to expand into the expanded state shown in FIGS. 6A and 6B.

In some embodiments, once released, the expanding section 510 may expand in volume. This expansion may cause the expanding section 510 to press outwardly against the tissue surfaces defining the gingival crevice. The resulting outward pressure may generate a wedging effect that may enhance the retention of the medicament seed 500 within the gingival crevice and hold the medicament seed 500 in place while the medicament section 520 releases the medicament (e.g., by dissolution or other mechanism) for absorption into the patient's system through the junctional epithelium. This retention is further enhanced by frictional forces between the surface of the expanded expanding section 510 and the gingival tissue walls. The secure placement achieved by this expansion and wedging may resist the displacement pressure within the gingival crevice that may otherwise displace the medicament seed 500 from the gingival crevice.

In some embodiments, the expanding section 510 may be made of a sponge-like material that is configured to conform to the shape and space of the gingival crevice and to fill the space while pressing against the space due to the sponge-like properties. For example, the elastic or restorative forces within the expanded sponge-like material of the expanding section 510, which resists compression, may exert an outwardly pressure against the surfaces of the gingival crevice may generate frictional forces creating a secure wedging effect that anchors the medicament seed 500 against the gingival crevice.

In some embodiments, the surface of the expanding section 510 may be configured with texture characteristics to increase friction and improve retention, and may be configured to create a “grippy” effect against the tissues of the gingival crevice.

In some embodiments, while the medicament section 520 may hold the primary medicament load, the expanding section 510 may also be configured to carry medicament. In embodiments, the medicament carried by the expanding section 510 may be the same medicament for extended release or a different type of medicament from the medicament carried by the medicament section 520. In some embodiments, the material of the sponge-like expanding section 510 may be configured to dissolve at a different rate (e.g., slower or faster) than the medicament section 520, allowing for multi-phasic release profiles.

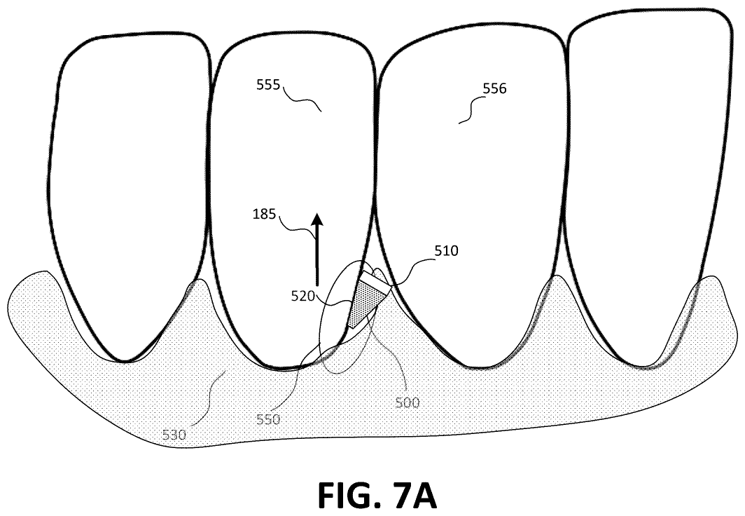

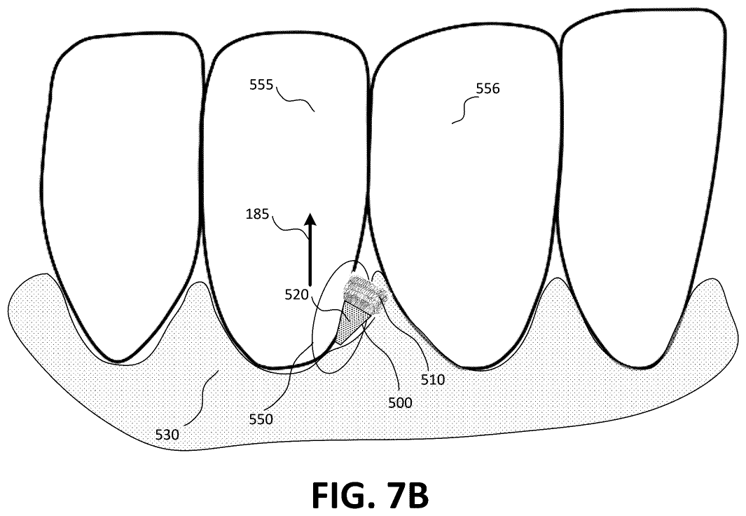

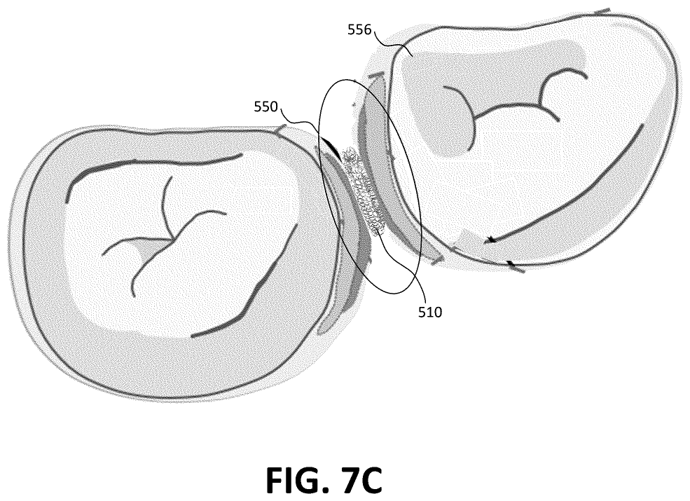

FIGS. 7A-7C illustrate an exemplary operational sequence for the deposition and retention of the medicament seed 500 within a patient's gingival crevice in accordance with embodiments of the present disclosure. This sequence demonstrates how the medicament seed 500, configured with the expanding section 510, may be used to achieve enhanced retention within the gingival crevice after deposition.

As shown in FIG. 7A, the medicament seed 500, in its pre-expansion compressed state may be deposited (e.g., using any deposition device) into the target gingival crevice 550. For example, as shown, the medicament seed 500 may be deposited into the gingival crevice 550 adjacent to tooth 555, within the gingival tissue 530. In particular, the medicament section 520 and the expanding section 510 may be deposited into the gingival crevice 550. At this stage, the expanding section 510 may still be in its compressed state, maintained in this state by the restraining mechanism 512.

Following the initial placement shown in FIG. 7A, the medicament seed 500 may be exposed to the moist environment within the gingival crevice 550. This exposure may initiate the activation of the retention mechanism. As described herein, the restraining mechanism 512 may degrade, dissolve, or otherwise release its constraint on the expanding section 510. As shown in FIG. 7B, the expanding section 510 may begin to expand within the gingival crevice 550. This expansion may cause the expanding section 510 to make contact with and exert outward pressure against the surrounding tissue walls of the gingival crevice 550. This outward pressure may counteract the natural tendency for displacement from the gingival crevice, indicated conceptually by arrow 185.

FIG. 7C illustrates a top view of the expanded state of the medicament seed 500 within the gingival crevice 550. As shown, the expanding section 510 has reached its substantially full expansion within the gingival crevice 550. The expanded expanding section 510 may conform to the shape of the gingival crevice 550 and may exert a sustained outward pressure, creating the wedging effect described herein. This secure anchoring may ensure the retention of the medicament seed 500 within the gingival crevice 550, allowing the medicament section 520 sufficient time to release the medicament payload for absorption into the patient's system through the junctional epithelium, while resisting displacement forces within the gingival crevice 550.

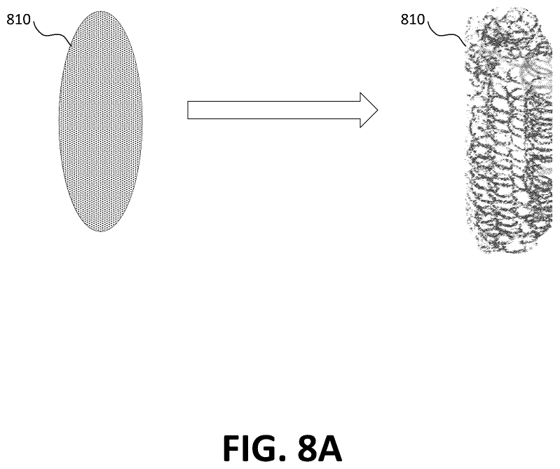

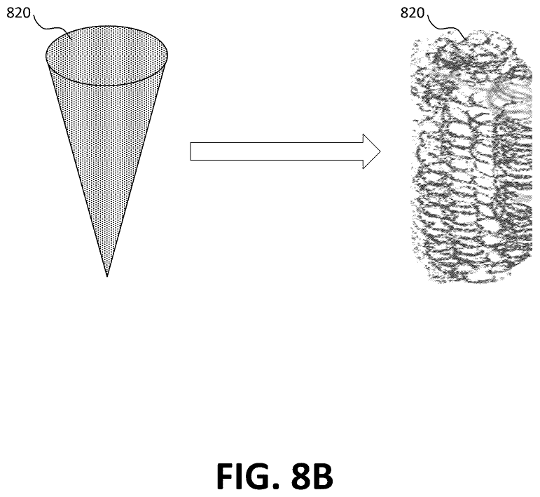

FIGS. 8A and 8B illustrate further embodiments of medicament seeds configured for enhanced retention within the gingival crevice in accordance with embodiments of the present disclosure. In embodiments, FIGS. 8A and 8B show that the overall form factor or shape of the medicament seed incorporating an expanding section may be varied while retaining the core retention mechanism.

For example, FIG. 8A shows an embodiment of a medicament seed 810. As shown, the medicament seed 810 may have a generally elongated shape, which may be, for example, cylindrical, oval, or somewhat bullet-shaped in profile. Similar to the embodiment described with reference to medicament seed 500, medicament seed 810 may include an expanding section and a medicament section or the entire medicament seed 810 may be an expanding section that carries the medicament (e.g., a single combined medicament section and expanding section). The expanding section may be configured to expand upon activation within the gingival crevice (e.g., via release from a compressed state by a restraining mechanism triggered by the environment within the gingival crevice) to provide retention through wedging and friction. The specific elongated geometry of medicament seed 810 may be advantageous for ease of insertion into certain gingival crevices or may provide a specific contact profile with the crevice walls upon expansion.

FIG. 8B shows another embodiment of a medicament seed 820, illustrating a different overall geometry. As shown, medicament seed 820 may have a generally arrow like conical, prism shape, or otherwise pointy shape, potentially possessing more defined edges compared to the embodiment of FIG. 8A. Medicament seed 820 may also operate based on the principle of an expanding section providing enhanced retention after deployment within the gingival crevice, analogous to medicament seed 500. The distinct shape of medicament seed 820 may offer different characteristics regarding stability, surface area contact with the gingival tissues upon expansion, or manufacturing considerations compared to the embodiments shown in FIG. 5A/5B or 8A.

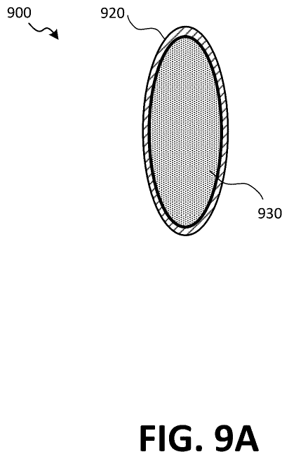

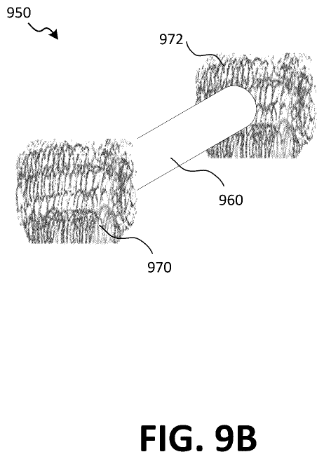

FIG. 9A shows another embodiment of a medicament seed 900 configured for enhanced retention within the gingival crevice in accordance with embodiments of the present disclosure. In this embodiment, the enhanced retention of the medicament seed 900 may be configured through application of a specialized coating applied to the exterior surface of the medicament seed 900's body.

For example, as shown, the medicament seed 900 may include a medicament carrier 930 and a coating 920. The medicament carrier 930 may be configured to contain, be made of, encapsulate, or otherwise carry the medicament intended for delivery into the patient's system through the junctional epithelium, and may be configured to dissolve or release the medicament over a period, as described herein. The coating 920 may applied to at least a portion of the external surface of the main body 930. In embodiments, the primary function of the coating 920 in this embodiment may be to interact with the tissues of the gingival crevice in a manner that increases the resistance of the medicament seed 900 to displacement forces, enhancing its retention time within the target site.

In embodiments, the specific configuration of the coating 920 may vary in different embodiments. For example, the coating 920 may include a bioadhesive or mucoadhesive material. Such materials may possess inherent properties that promote adhesion to the mucosal surfaces lining the gingival crevice upon contact. This adhesion may help to hold the medicament seed 900 securely in place against the tissue wall of the gingival crevice.

In additional or alternative embodiments, the coating 920 may be configured as a moisture-activated adhesive or glue. In these embodiments, the coating 920 may be relatively non-tacky prior to insertion, but upon exposure to the moisture present within the gingival crevice, the coating 920 may undergo a physical or chemical change that renders it adhesive. This activation mechanism ensures that the adhesive properties are present only after the medicament seed 900 has been deposited into the gingival crevice.

In some embodiments, the coating 920 may include surface features or characteristics configured to enhance mechanical retention. For example, the coating 920 may include a specific surface texture or include micro-features (such as micro-barbs, ridges, or grooves, analogous to surface features described in) that may increase frictional resistance or provide points of mechanical interlocking with the surrounding gingival tissue. In some embodiments, the coating 920 may include materials that exhibit controlled swelling upon hydration, increasing contact pressure and friction within the gingival crevice, or materials that may be configured to interact with components of saliva or gingival crevicular fluid to promote adhesion.

FIG. 9B shows yet another embodiment of a medicament seed 950 configured for enhanced retention within the gingival crevice in accordance with embodiments of the present disclosure. In this embodiment, the enhanced retention of the medicament seed 950 may be configured through a specialized form, which resembles a “dumbbell.” As can be seen, the medicament seed may include two expanding components 970 and 972 (similar to the expanding component 510 described with reference to FIGS. 5A-7C) connected by a shaft 960. This is an example of an implementation of a medicament seed in which the retaining features (e.g., the sponge-like material) may not be disposed along the entire length of the medicament seed, but rather just a portion. This seed may operate similar to the medicament seed described with reference to FIGS. 5A-7C but may include sponge-like material in the expanding components 970 and 972, while the medicament carrier 960 may carry the medicament.

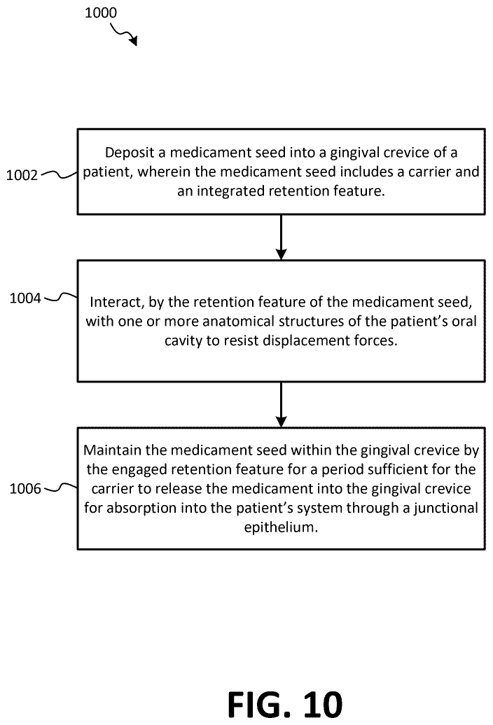

Operation for deposition and enhanced retention of a medicament seed will now be discussed with respect to FIG. 10 in accordance with embodiments of the present disclosure. FIG. 10 shows a high-level flow diagram 1000 of operations for a medicament seed configured with enhanced retention within the gingival crevice of a patient in accordance with embodiments of the present disclosure. In embodiments the operations of flow diagram 1000 may include operations for deposition and enhanced retention of a medicament seed configured as a medicament seed disclosed herein and as described with respect to FIGS. 1-9.

At block 1002, a medicament seed may be deposited into the gingival crevice of a patient. In embodiments, the medicament carrier may include a carrier and an integrated retention feature. In embodiments, the deposition may include utilizing a deposition device (e.g., as suggested in any of FIGS. 1-9 or any other deposition device that may be configured to deposit a medicament seed into a gingival crevice), or other suitable means to place the medicament seed within the potential space adjacent to the junctional epithelium 557. In embodiments, the insertion of the medicament seed may create a physical space within the gingival crevice.

At block 1004, the retention feature of the medicament seed interacts with one or more anatomical structures of the patient's oral cavity to resist displacement forces. In embodiments, the interaction between the retention feature of the medicament seed and the one or more anatomical structures of the patient's oral cavity may occur intrinsically upon placement, such as the wedging action of geometric configurations against the interdental papilla 560 or dental junction 561. In some embodiments, the interaction between the retention feature of the medicament seed and the one or more anatomical structures of the patient's oral cavity may include the activation of the retention feature upon exposure to the crevicular environment, such as the expansion of an expanding section or the activation of bioadhesive or moisture-activated coatings. In embodiments, the interaction between the retention feature of the medicament seed and the one or more anatomical structures of the patient's oral cavity may operate to anchor the medicament seed against natural expulsion or displacement forces within the gingival crevice.

At block 1006, the medicament seed is maintained within the gingival crevice by the engaged retention feature for a period sufficient for the carrier to release the medicament into the gingival crevice for absorption into the patient's system through a junctional epithelium. In embodiments, while retained, the carrier of the medicament seed may dissolve, degrade, or otherwise release its medicament payload into the localized space within the gingival crevice maintained by the seed. This medicament release in close proximity to the junctional epithelium may facilitate the absorption of the medicament into the patient's system through the junctional epithelium.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

Moreover, the description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f). Even under the broadest reasonable interpretation, in light of this paragraph of this specification, the claims are not intended to invoke 35 U.S.C. § 112(f) absent the specific language described above.

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the disclosures can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

What is claimed is:

1. A medicament seed configured for enhanced retention within a gingival crevice of a patient, comprising:
a carrier configured to be deposited into the gingival crevice and to carry a medicament for release within the gingival crevice to be absorbed into the patient's system through a junctional epithelium; and
a retention feature integrated with the carrier, wherein the retention feature is configured to interact with one or more anatomical structures of an oral cavity of the patient to resist displacement forces within the gingival crevice and maintain the carrier within the gingival crevice for a period sufficient for the release of the medicament into the gingival crevice.

2. The medicament seed of claim 1, wherein the retention feature includes a geometric configuration of the carrier configured to wedge against an anatomical structure within the oral cavity of the patient.

3. The medicament seed of claim 2, wherein the geometric configuration includes an A-frame structure having at least one leg and a vertex, wherein the vertex is configured to abut or wedge against a dental junction of an interdental papilla within the oral cavity of the patient, and wherein the at least one leg is configured to extend into the gingival crevice.

4. The medicament seed of claim 1, wherein the retention feature includes a surface modification on at least a portion of an exterior surface of the carrier.

5. The medicament seed of claim 4, wherein the surface modification includes microstructural features selected from the group consisting of texturing, micro-barbs, ridges, and grooves, configured to mechanically engage the gingival tissues.

6. The medicament seed of claim 4, wherein the surface modification includes a coating applied to the at least a portion of the exterior surface.

7. The medicament seed of claim 6, wherein the coating includes one or more of bioadhesive materials, moisture-activated adhesives, or combinations thereof.

8. The medicament seed of claim 1, wherein the retention feature includes an expanding section configured to volumetrically expand after deposition within the gingival crevice, the expansion causing the expanding section to exert outward pressure against tissue walls of the gingival crevice generating a wedging effect.

9. The medicament seed of claim 8, wherein the expanding section includes a sponge-like material held in a compressed state prior to deposition by a restraining mechanism, the restraining mechanism configured to release the sponge-like material upon exposure to the gingival crevice.

10. The medicament seed of claim 9, wherein the restraining mechanism includes a dissolvable material encapsulating or constraining the sponge-like material.

11. The medicament seed of claim 8, wherein the expanding section is further configured to carry a medicament.

12. The medicament seed of claim 1, wherein the retention feature includes the carrier configured as a flowable material including one or more of a gel and a highly viscous fluid, the flowable material configured to conform to the gingival crevice upon deposition.

13. The medicament seed of claim 12, wherein the flowable material is further configured to harden after a period following deposition within the gingival crevice.

14. A system for delivering medicament into one or more gingival crevices of a patient, the system comprising:
a medicament seed comprising a carrier configured to carry a medicament and a retention feature configured to resist displacement forces within the gingival crevice; and
a deposition device configured to facilitate placement of the medicament seed into the one or more gingival crevices.

15. The system of claim 14, wherein the deposition device comprises:
a handle;
an insertable portion configured to hold the medicament seed and be inserted proximate to or into the gingival crevice; and
a mechanism configured to release the medicament seed from the insertable portion upon placement.

16. The system of claim 16, wherein the mechanism configured to release the medicament seed includes one or more breakable tabs connecting the medicament seed to the insertable portion.

17. The system of claim 14, wherein the deposition device includes a spring-loaded deposition device, including:
an insertable portion configured to be inserted into the gingival crevice and comprising an opening;
an actuator configured to generate an expulsion force upon activation; and
a force transmission configured to apply the expulsion force to the medicament seed to expel the medicament seed through the opening and deposit the medicament seed into the gingival crevice.
